(19) United States
(12) Patent Application Publication
(10) Pub. No.: US 2017/0049705 A1
(43) Pub. Date: Feb. 23, 2017

(54) MONOLITHIC TABLETS BASED ON CARBOXYL POLYMERIC COMPLEXES FOR CONTROLLED DRUG RELEASE

(71) Applicant: MATRIPHARM INC., Laval (CA)

(72) Inventors: Mircea-Alexandru MATEESCU, Montreal (CA); Tien Canh LE, Montreal (CA)

(21) Appl. No.: 15/307,876

(22) PCT Filed: Apr. 28, 2015
(86) PCT No.: PCT/CA2015/000281

§ 371 Date: Oct. 31, 2016

Related U.S. Application Data
(60) Provisional application No. 61/985,772, filed on Apr. 29, 2014.

(51) Int. Cl. A61K 9/20; A61K 31/606; A61K 31/155

(52) U.S. Cl. CPC A61K 9/2054; A61K 31/155; A61K 31/606; A61K 9/2059

(57) ABSTRACT

The present document describes a dosage form for delivery of an active ingredient comprising: a first carboxylated polymer having carboxyl groups, having a degree of substitution of at least 0.2, a molecular weight of at least 200 kDa, and at least 30% of said carboxyl groups being complexed with a divalent cation; alone or in a co-complex with at least one of a) a control release polymer chosen from an insoluble polymer or a polymer having a reduced water solubility at 30° C., and a soluble polymer; and b) a second carboxylated polymer having carboxyl groups complexed with a divalent cation. The document also describes processes of making a carboxylated polymer having carboxyl groups, having a degree of substitution of at least 0.2, a molecular weight of at least 200 kDa, and at least 30% of said carboxyl groups being complexed with a divalent cation, and an inclusion complex, a co-complex, or both comprising the same.

### CROSS-REFERENCE TO RELATED APPLICATIONS

This application claims priority of U.S. provisional patent application 61/985,772 filed on Apr. 29, 2014, the specification of which is hereby incorporated by reference.

### BACKGROUND

(a) Field

The present invention relates to novel matrix obtaining by entrapment of at least a polymer in carboxyl polymers or by complexation of carboxyl polymers with multivalent metal ions (preferably calcium) and processes for its manufacture. Such complexes are useful as excipient for controlled-release of several drugs in a monolithic tablet dosage form.

(b) Related Prior Art

There are several drugs based on active agents which are highly soluble. These drugs are difficult to formulate due to their high solubility and considering the high doses recommended for administration. Metformin is such a high soluble oral anti-hyperglycemic drug currently used in the treatment of type 2 diabetes. Furthermore, the absorption window is mainly the upper part of intestine with an absolute bioavailability of about 60%. A relatively short plasma half-life of 1.5-4.5 h in combination with a rapid elimination up to 30% recovered in faeces explains the need of a better formulation. Its absorption is estimated to be complete within 6 h after administration and presumably confined to the upper intestine. In addition, Metformin has been shown to have a dose-dependent absorption suggesting some forms of saturable absorption or permeability/transit time-limited absorption.

An obstacle to a successful therapy with Metformin is the high incidence of concomitant gastrointestinal symptoms such as abdominal discomfort, vomiting, nausea, and diarrhea, etc. that can occur during treatment. Side effects represent an important barrier to successful treatment and the need for two or three doses per day, when high dosage is required, can lead to a decrease of patient compliance.

Side effects of Metformin could be minimized by using a controlled-dissolution (sustained-release) system. These technologies could prolong the duration of action of Metformin and improve patient compliance. There are several controlled-release systems currently commercialized in the market such as Glucophage SR® and Glumetza®. Most of these systems are based on expandable Gastro-Retentive Dosage Formulations (GRDF) which are easily swallowed but they are enlarged due to swelling thus prolonging their retention time in the stomach. Such GRDF are used for the release of Metformin mainly at the level of the stomach and of the upper intestine.

Although this GRDF technology has some advantages in terms of reduction of the number of daily dosing when compared with conventional immediate release formulations, no decrease of the gastro-intestinal side effects associated to Metformin extended-release forms were reported, probably due to a too long retention time of the tablets in the stomach (up to 8 h), Metformin release occurs locally and continuously in the stomach and in the upper intestine, which can induce a saturable absorption or permeability/transit time-limited absorption), high dose of drug required to achieve beneficial effects, incomplete release from the tablets due to the interactions of Metformin with the excipient that lead up to 8% loss of recovery. In this context, a possible bioaccumulation phenomenon could occur for patients requiring high dosage.

The present invention consists in monolithic systems compatible with various active pharmaceutical ingredients, especially for highly soluble drugs such as Metformin, Metoclopramide, Bupropion, Metoprolol, etc.

For these drugs and particularly for Metformin, this system appears as unique being able to limit the active principle ingredients (API) saturation and bioaccumulation phenomena, and is thus widely different from the systems used for GRDF. In addition, the new system releases Metformin not only in stomach and to upper intestine, but also in the whole gastrointestinal tract including the colon. This aspect is important, considering that Metformin can be absorbed about 40% in the remaining part of (lower) intestine tract.

Several excipients based on carboxylic polymers are currently used in the pharmaceutical formulations such as sodium carboxymethyl-cellulose (carmellose) or cross-linked sodium carboxymethyl-cellulose (Croscarmellose), sodium starch glycolate (Explotab), copolymer of methacrylic acid or divinylbenzene (polyacrilin potassium), etc. Other substances such as sodium bicarbonate in combination with citric or with tartaric acids, or sodium alginate at a low concentration, are also used. In general, these excipients are introduced in pharmaceutical formulations as disintegrating agent in order to deliver rapidly the active principle. In certain cases, excipients are also introduced in pharmaceutical formulations as diluting or binding agents, but none of these polymers are currently used as a principal excipient to control the drug release.

There are numerous natural polymers containing carboxylate groups such as alginate, hyaluronate or pectate, etc. Although these polymers present several interesting properties, they are expensive and it is difficult to control their hydration and then their subsequent swelling due to variations in their heterogenic structure. For example, alginate is a copolymer of β-D-mannuronic and α-L-guluronic acid residues. The variation of ratio and sequential distribution of mannuronic and guluronic acid residues along the chain length confers to alginate different mechanical and gelling properties. Similarly, hyaluronate is composed of D-glucuronic acid and N-acetyl-D-glucosamine residues, whereas pectate is composed of D-galacturonic acid and D-galacturonic acid methyl ester residues.

These carboxyl polymers (i.e. carboxymethyl-cellulose) are biologically and chemically inert, low cost to manufacture. It is possible at carboxylation to control the degree of substitution (DS) and thus degree of swelling.

For carboxymethylation, polysaccharides are activated in aqueous alkaline solution (mostly sodium hydroxide) and treated with monochloroacetic acid (or its sodium salt) to yield the carboxymethyl polysaccharide derivative.

Numerous carboxymethyl polymers such as carboxymethyl cellulose (Carmellose) or carboxymethyl starch (Explotab) currently commercialized in the market are used as additives in pharmaceutical formulations with different roles such as disintegrating, binding or diluent agents.

An important limitation of the use of carboxymethyl polymers as matrix for controlled or extended release is the presence of salts resulted from by-products (mainly sodium chloride and sodium glycolate when using monochloroacetate as carboxylation agent).

### SUMMARY

According to an embodiment, there is provided a dosage form for delivery of an active ingredient comprising:
a first carboxylated polymer having carboxyl groups, having
   a degree of substitution of at least 0.2,
   a molecular weight of at least 200 kDa, and
   at least 30% of the carboxyl groups being complexed with a divalent cation;
in a co-complex, entrapped within, or both, with at least one of
   a control release polymer chosen from
      an insoluble polymer or a polymer having a reduced water solubility at 30° C.,
      a soluble polymer; and
   a second carboxylated polymer having carboxyl groups complexed with a divalent cation.

The divalent cation may be chosen from calcium, magnesium, zinc, aluminum, copper, or combinations thereof.

The divalent cation may be calcium.

The control release polymer may have a molecular weight equal to or smaller than the molecular weight of the first carboxylated polymer.

The second carboxylated polymer having carboxyl groups complexed with a divalent cation may be having
a degree of substitution of at least 0.15,
a molecular weight equal to or smaller than the molecular weight of the first carboxylated polymer, and
at least 50% of the carboxyl groups being complexed with a divalent cation.

The first or second carboxylated polymer may be chosen from a carboxymethylcellulose, a carboxymethyl starch, a carboxymethyl high amylose starch, a carboxyethyl starch, a carboxyethyl high amylose starch, a succinyl-starch, a succinyl high amylose starch, a carboxymethyl chitosan, a carboxyethyl chitosan, a succinyl chitosan, a carboxymethyl guar gum, a carboxymethyl hydroxypropyl guar gum, a gellan gum, a xanthan gum, a alginate, a pectate, a hyaluronate, a polyacrylic acid, a polymethacrylic acid, a copolymer of acrylic and methacrylic acids, or combination thereof.

The first carboxylated polymer may be carboxymethylcellulose.

The second carboxylated polymer may be carboxymethyl starch.

The insoluble polymer or polymer having a reduced water solubility at 30° C. may be chosen from a cellulose, a methylcellulose, an ethylcellulose, an ethylmethylcellulose, an hydroxyethyl-cellulose, an hydroxyethylmethylcellulose, an ethyl hydroxyethylcellulose, a propylcellulose, an hydroxypropylcellulose, an hydroxypropylmethylcellulose, a starch, a hydroxypropylstarch, a starch acetate, a cross-linked starch, an agar, an agarose, a guar, an hydroxypropylguar, a pullulan, a carrageenan, a scleroglucan and combinations thereof.

The insoluble polymer or polymer having a reduced water solubility at 30° C. may be a methylcellulose, an ethylcellulose, and an hydroxypropylmethylcellulose, or combinations thereof.

The soluble polymer may be chosen from a polyvinylalcohol, a polyethyleneglycol, polycaprolactone, a polyvinyl-pyrrolidone, and combinations thereof.

The ratio of the first carboxylated polymer and the control release polymer, the second carboxylated polymer, or a combination thereof, may be from about 1:1 to 90:10 w/w.

The ratio of the first carboxylated polymer and the control release polymer, the second carboxylated polymer, or a combination thereof, may be about 60:40 w/w.

The ratio of the first carboxylated polymer and the control release polymer, the second carboxylated polymer, or a combination thereof, may be about 70:30 w/w.

The ratio of the first carboxylated polymer and the control release polymer, the second carboxylated polymer, or a combination thereof, may be about 90:10 w/w.

The ratio of the first carboxylated polymer and the control release polymer, the second carboxylated polymer, or a combination thereof, may be about 1:1 w/w.

The molecular weight of the control release polymer or the second carboxylated polymer may be from about 15 kDa to about 200 kDa.

The molecular weight of the control release polymer or the second carboxylated polymer may be from about 15 kDa to about 80 kDa.

The degree of substitution of the first carboxylated polymer may be from about 0.2 to about 2.

The degree of substitution of the first carboxylated polymer may be from about 0.2 to about 0.9.

The degree of substitution of the first carboxylated polymer may be from about 0.3 to about 0.9.

The degree of substitution of the first carboxylated polymer may be from about 0.3 to about 0.7.

The degree of substitution of the first carboxylated polymer may be from about 0.3 to about 0.5.

The degree of substitution of the first carboxylated polymer may be about 0.5.

The degree of substitution of the second carboxylated polymer may be from about 0.2 to about 2.

The degree of substitution of the second carboxylated polymer may be from about 0.2 to about 1.

The degree of substitution of the second carboxylated polymer may be from about 0.3 to about 0.9.

The degree of substitution of the second carboxylated polymer may be from about 0.3 to about 0.7.

The degree of substitution of the second carboxylated polymer may be from about 0.3 to about 0.5.

The degree of substitution of the second carboxylated polymer may be about 0.5.

The first carboxylated polymer may be carboxymethyl cellulose having degree of substitution of about 0.5.

The first carboxylated polymer may be carboxymethyl starch having degree of substitution of about 0.5.

The dosage for may be further comprising the active ingredient.

The active ingredient may be chosen from a highly soluble drug, or a drug having low solubility.

The highly soluble drug may be chosen from metformin, acyclovir, alendronate, atenolol, bupropion, captopril, cinnarizine, ciprofloxacin, cisapride, ganciclovir, g-csf, glipizide, ketoprofen, levodopa, melatonin, metoclopramide, metoprolol, minocyclin, misoprostol, nicardipine, riboflavin, sotalol, tetracycline, and verapamil.

The highly soluble drug may be metformin.

The drug having low solubility may be chosen from diclofenac, sulfasalazine, prednisone, azathioprine, metronidazole, ampicillin, ciprofloxacin, cephalosporin, furosemide, tetracycline, sulfonamide, mesalamine, acetylsalicylic acid, irbesartan, lisinopril, rabeprazole, sertraline, simvastatin, pioglitazone, paroxetine, terbinafine, valproic, venlafaxine, atorvastatin, bicalutamide, citalopram, fluoxetine, supeudol, pravastatin, diltiazem, and bupropion.

The drug having low solubility may be mesalamine.

The highly soluble drug may be from about 500 mg to about 1200 mg metformin, the first carboxylated polymer having carboxyl groups may be carboxymethyl cellulose and the control release polymer may be methylcellulose.

The drug having low solubility may be from about mg 400 to about 1000 mg mesalamine, the first carboxylated polymer having carboxyl groups may be carboxymethyl cellulose and the control release polymer may be methylcellulose.

According to another embodiment, there may be provided a method of treating diabetes comprising administering to a subject in need thereof a dosage form of the present invention.

According to another embodiment, there may be provided a method of treating an inflammatory bowel disease comprising administering to a subject in need thereof a dosage form of the present invention.

According to another embodiment, there may be provided a use of the dosage form of the present invention for treating diabetes.

According to another embodiment, there may be provided a use of the dosage form of the present invention for treating an inflammatory bowel disease.

According to another embodiment, there may be provided a dosage form of the present invention for use in the treatment of diabetes.

According to another embodiment, there may be provided a dosage form of the present invention for use in the treatment of an inflammatory bowel disease.

According to another embodiment, there may be provided a process for the preparation of a carboxylated polymer having carboxyl groups complexed with a divalent cation comprising:
a) in a solution at pH>5.5, contacting a carboxylated polymer having carboxyl groups, having a degree of substitution of at least 0.2, and a molecular weight of at least 200 kDa with a source of divalent cation for a time sufficient for at least 50% of the carboxyl groups to be complexed with the divalent cation.

The process may be further comprising step b):
b) precipitating the carboxylated polymer having carboxyl groups complexed with a divalent cation, to obtain a precipitated carboxylated polymer having carboxyl groups complexed with a divalent cation.

The process may be further comprising step c):
b) collecting and drying the precipitated carboxylated polymer having carboxyl groups complexed with a divalent cation.

The source of divalent cation may be calcium chloride, calcium lactate, calcium acetate, calcium gluconate, and combinations thereof.

According to another embodiment, there may be provided a process for the preparation of an inclusion complex, a co-complex, or both comprising:
a) contacting a solution at pH>5.5, containing
   a first carboxylated polymer having carboxyl groups, having a degree of substitution of at least 0.2, and a molecular weight of at least 200 kDa, and
   at least one of
   a control release polymer chosen from
      an insoluble polymer or a polymer having a reduced water solubility at 30° C.,
      a soluble polymer; and
   a second carboxylated polymer having carboxyl groups complexed with a divalent cation.
with a source of divalent cation for a time sufficient for at least 50% of the carboxyl groups to be complexed with the divalent cation.

The divalent cation may be chosen from calcium, magnesium, zinc, aluminum, copper, or combinations thereof.

The divalent cation may be calcium.

The second carboxylated polymer having carboxyl groups complexed with a divalent cation may be having
a degree of substitution of at least 0.15,
a molecular weight equal to or smaller than the molecular weight of the first carboxylated polymer, and
at least 50% of the carboxyl groups being complexed with a divalent cation.

The first or second carboxylated polymer may be chosen from a carboxymethylcellulose, a carboxymethyl starch, a carboxymethyl high amylose starch, a carboxyethyl starch, a carboxyethyl high amylose starch, a succinyl-starch, a succinyl high amylose starch, a carboxymethyl chitosan, a carboxyethyl chitosan, a succinyl chitosan, a carboxymethyl guar gum, a carboxymethyl hydroxypropyl guar gum, a gellan gum, a xanthan gum, a alginate, a pectate, a hyaluronate, a polyacrylic acid, a polymethacrylic acid, a copolymer of acrylic and methacrylic acids, or combination thereof.

The first carboxylated polymer may be carboxymethylcellulose.

The second carboxylated polymer may be carboxymethyl starch.

The insoluble polymer or polymer having a reduced water solubility at 30° C. may be chosen from a cellulose, a methylcellulose, an ethylcellulose, an ethylmethylcellulose, an hydroxyethyl-cellulose, an hydroxyethylmethylcellulose, an ethyl hydroxyethylcellulose, a propylcellulose, an hydroxypropylcellulose, an hydroxypropylmethylcellulose, a starch, a hydroxypropylstarch, a starch acetate, a cross-linked starch, an agar, an agarose, a guar gum, an hydroxypropylguar, a pullulan, a carrageenan, a scleroglucan and combinations thereof.

The insoluble polymer or polymer having a reduced water solubility at 30° C. may be a methylcellulose, an ethylcellulose, and an hydroxypropylmethylcellulose, or combinations thereof.

The soluble polymer may be chosen from a polyvinylalcohol, a polyethyleneglycol, polycaprolactone, a polyvinyl-pyrrolidone, and combinations thereof.

The first carboxylated polymer and the control release polymer, the second carboxylated polymer, or a combination thereof, may be from about 1:1 to 90:10 w/w.

The ratio of the first carboxylated polymer and the control release polymer, the second carboxylated polymer, or a combination thereof, may be about 60:40 w/w.

The ratio of the first carboxylated polymer and the control release polymer, the second carboxylated polymer, or a combination thereof, may be about 70:30 w/w.

The ratio of the first carboxylated polymer and the control release polymer, the second carboxylated polymer, or a combination thereof, may be about 90:10 w/w.

The ratio of the first carboxylated polymer and the control release polymer, the second carboxylated polymer, or a combination thereof, may be about 1:1 w/w.

The molecular weight of the control release polymer or the second carboxylated polymer may be from about 15 kDa to about 200 kDa.

The molecular weight of the control release polymer or the second carboxylated polymer may be from about 15 kDa to about 80 kDa.

The degree of substitution of the first carboxylated polymer may be from about 0.2 to about 2.

The degree of substitution of the first carboxylated polymer may be from about 0.2 to about 0.9.

The degree of substitution of the first carboxylated polymer may be from about 0.3 to about 0.9.

The degree of substitution of the first carboxylated polymer may be from about 0.3 to about 0.7.

The degree of substitution of the first carboxylated polymer may be from about 0.3 to about 0.5.

The degree of substitution of the first carboxylated polymer may be about 0.5.

The degree of substitution of the second carboxylated polymer may be from about 0.2 to about 1.0.

The degree of substitution of the second carboxylated polymer may be from about 0.2 to about 0.9.

The degree of substitution of the second carboxylated polymer may be from about 0.3 to about 0.9.

The degree of substitution of the second carboxylated polymer may be from about 0.3 to about 0.7.

The degree of substitution of the second carboxylated polymer may be from about 0.3 to about 0.5.

The degree of substitution of the second carboxylated polymer may be about 0.5.

The first carboxylated polymer may be carboxymethyl cellulose having degree of substitution of about 0.5.

The first carboxylated polymer may be carboxymethyl starch having degree of substitution of about 0.5.

The process may be further comprising step b):
b) precipitating the inclusion complex, the co-complex, or both, to obtain a precipitated inclusion complex, a precipitated co-complex, or both complex.

The process may be further comprising step c):
c) collecting and drying the precipitated inclusion complex, the precipitated co-complex, or both.

The following terms are defined below.

As used herein, the term <<functionalizing starch>> or <<functionalized starch>> is intended to mean functionalization that is not limited to the conversion of the native or modified starch by carboxymethylation, but also includes possible functionalization of other starch derivatives such as starch succinate (succinyl starch), hydroxypropyl starch, acetyl starch, hydroxypropyl methyl starch, acid modified starch, octenyl starch, pregelatinized starch or mixture thereof.

The term <<functionalization>> as used herein is intended to mean the addition by covalent bonds of carboxyl groups (or its derivatives) onto the starch chains. The functionalization can be (but is not limited to) the carboxylation (addition of carboxylate groups), amination (addition of amine groups), alkylation (addition of alkyl groups) or acylation (addition of acyl groups).

The term <<carboxylation>> as used herein is intended to mean the addition of carboxyl groups onto the polysaccharide macromolecule. Possible carboxylation includes but not limited to the carboxymethylation, carboxyethylation, succinylation, acrylation, etc. According to a preferred embodiment, the carboxylation is a <<carboxymethylation>>.

The term <<degree of substitution>> is intended to mean the average number of substituents per glucose unit (GU), the monomer unit of starch. Since each GU contains three hydroxyl groups, the DS can vary between 0-3. According to an embodiment of the present invention, the DS may be equal to or greater than 0.2 such as to obtain for certain BA up to 80% (w/w) incorporated in the functionalized carboxyl polymer (e.g. CMS).

The term <<bioactive agent>> or <<active agent>> or <<active ingredient>> is intended to mean compounds or mixtures thereof having or producing an effect on living organisms. Examples include particularly metformin, acyclovir, alendronate, atenolol, bupropion, captopril, cinnarizine, ciprofloxacin, cisapride, ganciclovir, g-csf, glipizide, ketoprofen, levodopa, melatonin, metoclopramide, metoprolol, minocyclin, misoprostol, nicardipine, riboflavin, sotalol, tetracycline, verapamil, diclofenac, sulfasalazine, prednisone, azathioprine, metronidazole, ampicillin, ciprofloxacin, cephalosporin, furosemide, tetracycline, sulfonamide, mesalamine, acetylsalicylic acid, irbesartan, lisinopril, rabeprazole, sertraline, simvastatin, pioglitazone, paroxetine, terbinafine, valproic, venlafaxine, atorvastatin, bicalutamide, citalopram, fluoxetine, supeudol, pravastatin, diltiazem, and bupropion.

The term “entrapment” is intended to mean the process by which the first carboxylated polymer having carboxyl groups is mixed with one or more additional polymers before being contacted with the source of the divalent cation. The complexation reaction with the carboxyl groups and divalent cations is performed in the presence of the one or more additional polymers, such that the one or more additional polymers is entrapped within the first carboxylated polymer having carboxyl groups, thereby forming an inclusion complex. The first carboxylated polymer is stabilized by the divalent cations. The one or more additional polymer entrapped within the first carboxylated polymer may or may not have specific interaction with the first carboxylated polymer stabilized with the divalent cations. See for example FIG. 3.

The term “co-complexation” is intended to mean the process by which the first carboxylated polymer having carboxyl groups is mixed with one or more additional polymers before being contacted with the source of the divalent cation. The complexation reaction with the carboxyl groups and divalent cations is performed in the presence of the one or more additional polymers, such that the one or more additional polymers is complexed with the first carboxylated polymer having carboxyl groups, thereby forming a “co-complex”. The first carboxylated polymer is stabilized by the divalent cations, and the one or more additional polymers can contribute to the stabilization of the co-complex. The one or more additional polymer co-complexed within the first carboxylated polymer may or may not have specific interaction with the first carboxylated polymer stabilized with the divalent cations. See for example FIG. 4.

The term <<composition>> as used herein is intended to encompass a product comprising the specified ingredients in the specified amounts, as well as any product which results, directly or indirectly, from combination of the specified ingredients in the specified amounts. Such term in relation to pharmaceutical composition or other compositions in general, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions or other compositions in general of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof.

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1: X-Ray diffraction of native and carboxymethyl starches. The crystalline structure of native starch presents a B-form pattern, whereas Carboxymethyl-starch presents a V-form organization;

FIG. 2: Complexation of Carboxymethyl cellulose with calcium ions;

FIG. 3: Schematical presentation of the <<Calcium Carboxymethyl-cellulose/Methyl-cellulose>> complex entrapment of Methyl-cellulose in Carboxymethyl-cellulose by complexation with calcium ions;

FIG. 4: Schematically presentation of Carboxymethyl-cellulose (CMC) Carboxymethyl-starch (CMS) Co-complexation of CMC/CMS with calcium ions;

FIG. 5: FTIR spectra of methyl-cellulose (MC), sodium (Na-CMC) and calcium (Ca-CMC) carboxymethyl-cellulose (CMC) and of Calcium carboxymethyl-cellulose/Methyl-cellulose (Ca-CMC/MC);

FIG. 6: FTIR spectra of complex calcium carboxymethyl-cellulose/Methylcellulose (Ca-CMC/MC) at various CMC/MC ratios;

FIG. 7: Schematical presentation of the hypothetical mechanism of Metformin controlled release from the complex Ca-CMC/MC as matrix;

FIG. 8: Metformin dissolution profiles of monolithic tablets at different ratios of Ca-CMC and MC compared with control articles. Ca-CMC/MC matrix-1 (ratio 60:40) and matrix-2 (ratio 70:30);

FIG. 9: Dissolution profiles of Metformin from monolithic tablets with MC of various molecular weights (15 kDa and 80 kDa) entrapped in Ca-CMC;

FIG. 10: Schematical presentation of the supposed of Metformin controlled release according to the molecular weights of MC entrapped in Ca-CMC;

FIG. 11: Pharmacokinetic profiles of Metformin (500 mg) formulated as Ca-CMC/MC Monolithic Tablets compared with commercial GRDF tablets in in vivo study on Beagle dogs;

FIG. 12: Cumulative area under the curve (AUC₀₋₂₄) of Glumetza® and Ca-CMC/MC monolithic tablet;

FIG. 13: FTIR spectra of sodium (Na) and calcium (Ca) carboxymethyl-starch and of calcium carboxymethyl-cellulose/carboxymethyl-starch (Ca-CMC/CMS) complexes at different CMC/CMS ratios;

FIG. 14: FTIR spectra of sodium (Na) and calcium (Ca) carboxymethyl-starch and of calcium carboxymethyl-starch and polyacrylic acids (Ca-CMS/PAA) complexes;

It will be noted that throughout the appended drawings, like features are identified by like reference numerals.

### DETAILED DESCRIPTION

In the present invention, a novel matrix type was developed based on carboxyl polymers complexed with divalent metal ions (i.e. calcium) and/or by entrapping another polymer. This novel matrix type is useful as excipient for controlled-release of several drugs, particularly highly soluble drugs (i.e. Metformin) in a monolithic tablet dosage form.

Generally, the carboxyl polymers currently commercialized possess a great capacity of hydration leading rapidly to the disintegration of solid dosage forms. The mechanism is due to the presence of anionic carboxyl forms (—COO⁻) and mobile counter-ions which attract more water to penetrate inside of the carboxyl polymer. The most common form of carboxyl polymer is the sodium form, where the carboxylate anion is balanced by a sodium counter-ion (Na⁺).

This high capacity of hydration induces a swelling of excipient and a fast release of the active principle. This is why these polymers cannot be used as the main excipient for the controlled or extended drug release.

In addition, the impurities and the by-products are mostly salts that are a powerful hydrating factor. These by-products are principally sodium chloride and sodium glycolate and a crude carboxymethyl cellulose technical product can contain up to 40% salts. The presence of sodium allows to shield the charge of the carboxylate groups and counteracts the repulsion that the ionized carboxylate groups exert on each other. The increase of ionic strength is an important factor favoring the penetration of water inside of polymers. At higher salt concentration, the polymer hydration capacity is stronger.

For these reasons, numerous carboxymethyl derivatives such as carboxymethyl cellulose, carboxymethyl starch or starch glycolate are used in tablets mostly as disintegrating agent and in certain case, as binder or diluting agents. In fact, the carboxymethyl polymers are generally able to form a gel network with weak immobile charge (—COO⁻). However, in the presence of salts (sodium chloride and sodium glycolate), the mobile counter-ion sodium of carboxylate (—COO⁻ Na⁺) could attract more water to penetrate inside the gel network inducing a swelling of tablet and rapid release the active principle.

High capacity of hydration also has a bad effect for solid dosage form such as expansion of tablet or loss of integrity (disintegration). For use as matrix for controlled drug release, it is necessary to reduce or eliminate salt and counter-ion (from anionic carboxylate) forms. The removal of salts can reduce the hydration which has an important impact on kinetic profiles of the drug controlled release.

The protonation of carboxyl groups improves the drug controlled release profiles. In fact, the protonated carboxylic acid form is resistant to low pH of the stomach, but then will start to break down at a pH of 6.5 and above. For this reason, the protonated carboxyl polymers were proposed as excipient for drug delayed delivery system (target delivery or chronodelivery) or as material for coating the tablets. They are often known as the pH-sensitive or pH-dependent systems.

However, it has been found that the pH-dependent systems present a high variability of drug release kinetic profiles. This behavior has been investigated by FDA and it has been found that when tablets failed to display the desired dissolution profile, this could be linked directly to the variability in the coating thickness of the wall around the tablet. Furthermore, this phenomenon could be influenced by the variation of the gastric acidity strength between individuals. For example, the pH value in the stomach in fasted healthy adult humans usually lies in the range pH 1-2. However, higher pH can be observed in elderly subjects due to waning ability to produce gastric acid. On the other hand, gastric pH can be increased by pharmacological interventions such as H₂— receptor antagonists (i.e. Cimetidine) or proton pump inhibitors (Omeprazole), which are widely used. Additionally, hyper-secretion of acid is not rare, mostly associated with specific diseases such as Zollinger-Ellison syndrome. After meal intake, pH in the stomach usually rises due to buffering effects of the meal contents, and may initially reach values>5.0, depending on meal composition.

The degree of substitution (DS) is also a critical parameter that can influence the kinetic profiles of API delivery.

In order to eliminate the sodium carboxylate form and improve the stability of carboxyl polymers in biological fluids, the complexation of carboxyl groups with multivalent cations (preferably calcium) is proposed.

The present invention comprises complexing carboxyl polymers or copolymers with multivalent cations permitting to obtain stable polymers which can be used as excipients for monolithic tablet for controlled drug delivery. Such complexation can stabilize carboxylic chains and reduce availability of carboxylate groups to interact with drugs.

The main advantages of this novel matrix are:
low or no interactions between matrix and active ingredients;
independent of or low sensitivity to different pH values during the passage through gastric (pH<2.0) and intestinal (pH≧6.8) tracts limiting the variability in dissolution;
versatile and compatible with various API;
excipient easy to produce;
simple to manufacture by direct compression of dried powders (no required special equipment);
high loading capacity of tablets formulations;
inexpensive and generally recognized as safe (GRAS) raw materials.

The characteristics of calcium complex of carboxymethyl polymers may depend on several factors such as:
degree of substitution (DS, number of carboxylate groups on the polymer);
state (charged or uncharged) of carboxylate groups;
molecular weight and structure of polymers.
   i) Degree of Substitution (Number of Carboxylate Groups on the Polymers which are Low Cost and Easy to Control the Substitution)

In case of cellulose or starch as model polymers, several degrees of carboxymethylation (in the range 0.05-1.0) were prepared in alkaline medium (NaOH 40%) through a nucleophilic substitution of polysaccharide (i.e. cellulose, 10 g) and sodium monochloroacetate (5.0-50 g) as described by Salmi et al. (Salmi, T., Valtakari, D., Paatero, E. 1994. Ind. Eng. Chem. Res., 33, 1454-1459) with slight modifications. The carboxymethyl cellulose obtained at various DS was then treated following the disclosure in excess of calcium chloride for at least 1 h at 50° C. under stirring in order to complex the calcium by carboxyl groups of carboxymethyl-cellulose. Finally, the solution containing the complex was precipitated and dried with pure acetone to obtain the corresponding derivative powders. For stability assay, monolithic tablets of 400 mg were obtained by direct compression of excipient powders and incubated at 37° C. under stirring (100 rpm/min) during 2 h in simulated gastric fluid (SGF, pH 1.5) and then in simulated intestinal fluid (SIF, pH 7.2).

The carboxymethyl-cellulose tablets with DS<0.3 were gradually eroded and disintegrated in SGF, whereas those with DS≧0.3 remained stable in SGF. Best results were obtained for tablets based on calcium carboxymethyl cellulose with DS about of 0.5 which were slightly swollen, but remained stable in SGF and SIF.

Similar results were obtained for carboxymethyl-starch. Indeed, the calcium carboxymethyl-starch tablets were remained stable in SGF and SIF for DS≧0.2.

It appears that there is a critical DS required to stabilize the matrix via complexation with calcium ions and this DS can vary for each type of polymer.

### ii) Charged or Protonated State of Carboxylate Groups on the Polymer:

The protonation of carboxyl groups (carboxylic acid, —COOH) generates stable gels that are less soluble than the polymer under carboxylate forms. In fact, carboxylate groups at low pH values (<3.0) are protonated. This uncharged form can contribute to the polymer stabilization by hydrogen associations generating a structure more stable, limiting thus the hydration. At pH values higher than 7.0, the carboxyl groups are mostly deprotonated (—COO⁻ Na⁺) under anionic form containing sodium mobile counter-ions favoring the hydration and swelling of the polymer.

When carboxymethyl cellulose (DS about of 0.5) are complexed with the calcium ions at various pH values (3.0-8.0), all tablets are stable in SGF and no significant swelling differences are noticed. However, different phenomena in SIF are observed for complexation at:

pH>5.5, tablets were further swollen, but remained stable. No visible erosion was observed after 6 h in SIF;

pH≦5.5, tablets were slightly swollen, but rapidly degraded by erosion after 6 h in SIF;

The explanation of these phenomena is based on the ionization of the carboxyl groups. Only carboxyl groups under anionic (—COO⁻, negative charge) forms possess a great affinity to complex with calcium. When the complexation occurred at pH values>5.5, most of carboxyl groups under anionic forms were generated (pKa of carboxyl is about of 4.8) in the medium. Consequently, there are more carboxylate groups available for complexation with calcium, and the stabilization of polymer in dissolution media was more important.

For the complexation with calcium at pH values <5.5, there is a protonation as carboxylic acid forms (uncharged, —COOH) with a lesser availability to complex calcium and the stability of polymer was weaker leading to a rapid degradation in SIF.

These analyses indicate that at higher availability of anionic forms (carboxylate groups) for complexation with calcium, the stability of tablets is increased. According to an embodiment, at least 30% of the carboxyl groups being complexed with a divalent cation. According to an embodiment, at least 40%, or 50%, or 60%, or 70%, or 80%, or 90% of the carboxyl groups being complexed with a divalent cation.

iii) Molecular Weight of Polymers which are Low Cost and Easy to Control the Substitutions

Carboxymethyl-cellulose (DS between 0.5-0.7) with different molecular weights (100-700 kDa) have been complexed with calcium and dried in pure acetone to obtain complexes calcium carboxymethyl-cellulose. At higher molecular weight of polymer, the rheological (including viscosity) properties are increased with a reduced hydration capacity. These powders are compressed to obtain tablets of 400 mg which are incubated for 2 h in SGF and then in SIF, as described previously.

All calcium carboxymethyl cellulose tablets are slightly swollen in SGF with the formation of a transparent gel layer around the tablet. The tablets based on calcium carboxymethyl cellulose with MW<200 kDa are characterized by a soft and sticky gel degraded in about 6 h by erosion in SIF, whereas those with MW≧200 kDa presented a compact gel, non-adhesive, swollen compared with its original size and stable in SIF over 24 h.

It was found for high molecular weights, an increased stability of tablets in SGF and SIF. Therefore, according to an embodiment, the molecular weight of the first carboxylated polymer having carboxyl groups may be at least 200 kDa.

iv) Structure of the Polymers which are Low Cost and Easy to Control the Substitution

Polymers can be stabilized in different ways generating several particular structures. For example, high amylose starch (α-1,4 linkages of glucose repeated units), a disordered amorphous conformation can co-exist with two different helical forms: simple helix (V-form) or double helix (A or B organization). The difference between the A and B forms is in the unit cell hydration of the crystalline structure.

Unlike the starch, cellulose is a straight chain polymer and no helix coiling or branching occurs. The molecule adopts an extended and rather stiff rod-like conformation due to the 6-1,4 linkages of every glucose unit in cellulose which is alternatively flipped promoting intra- and inter-chain hydrogen bonds, as well as Van der Waals interactions. These associations make cellulose linear and highly crystalline.

Generally, cellulose and starch possess particular structures which are organized, insoluble in water and majority of solvents. However, the solubility may be enhanced by carboxymethylation.

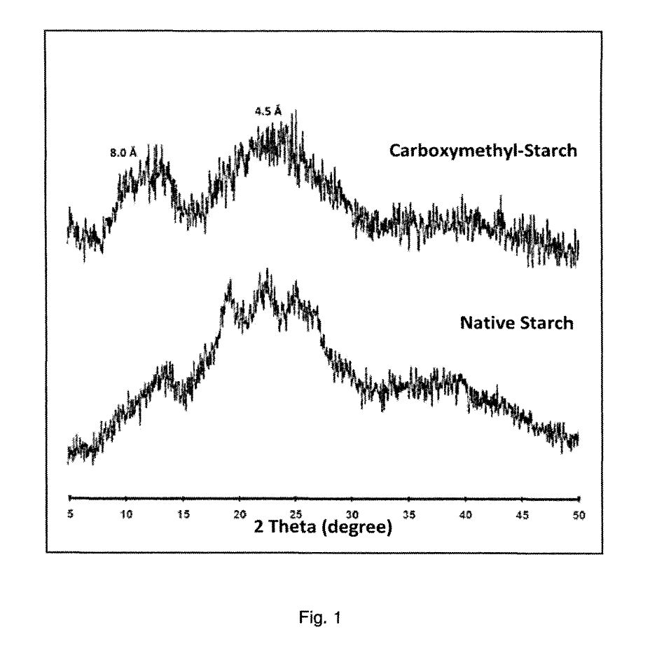

When cellulose is functionalized by addition carboxymethyl groups using monochloroacetate as described previously, its crystalline structure is altered and generated an amorphous structure in several parts of the polymer. This phenomenon could be observed by X-ray diffraction analysis (FIG. 1) and the alteration is probably due to the carboxyl groups which are inserted between macromolecular chains preventing hydrogen interactions and decreasing thus the crystallinity of cellulose.

Similar observations are noticed for starch when it is carboxymethylated in the same conditions (pH, temperature, degree of substitution, etc.). Following the method discussed above, these carboxymethyl polymers are treated in excess of calcium chloride solution to form complexes and then dried with pure acetone to obtain the corresponding powders. Also, the source of calcium cations may be calcium chloride, calcium lactate, calcium acetate, calcium gluconate, and combinations thereof

Dissolution tests are carried out during 2 h in SGF followed SIF using carboxymethyl-cellulose or carboxymethyl-high amylose starch as excipients (tablets of 400 mg obtained by direct compression 2.3 T/cm²).

The results show that calcium carboxymethyl-starch tablets are swollen with a gel-like structure occurring around the tablet, which remained stable in SGF and in SIF.

For calcium carboxymethyl-cellulose tablets, a moderate swelling in SGF is observed. In SIF, they are more inflated than carboxymethyl-starch (CMS) tablets, but still stable up to 24 h.

The explanation of the calcium carboxymethyl-cellulose (CMC) higher swelling in SIF may be due to the linear structure of cellulose which allows to reactants a better accessibility to the OH groups. Consequently, due to the carboxymethyl groups, the crystalline cellulose structure is moderately altered and the stabilization by complexation with calcium is more important than intermolecular hydrogen interactions.

When comparing the structure of cellulose with that of high amylose starch (HAS), the accessibility at hydroxyl group sites of HAS is probably hindered due to the helical structure. According to the X-ray analysis, the helical structure of starch (B-form) is altered by carboxymethylation and changed to the V-form.

After carboxymethylation, the hydrogen associations of starch helical structure of CMS are less stable than those of cellulose linear structure. This observation can explain why for carboxymethyl-cellulose is required a DS (>0.30) higher than that of carboxymethyl-starch (DS>0.15) to generate a stable complex with calcium.

In order to obtain a stable matrix in SGF and SIF, the carboxyl polymers generally must possess:
A certain degree of substitution required to generate a stable structure when complexed with multivalent cations. This DS can vary according for each type of polymer;
A pH value for the complexation with multivalent cations superior to pKa value of carboxyl groups of polymer. The pH value for the complexation ensures that at least 30% of the carboxyl groups are complexed with a multivalent cation, and has the desired properties.
A relatively high molecular weight;

According to an embodiment, the DS may be from about 0.2 to about 3, or from about 0.2 to about 2.5, or from about 0.2 to about 2, or from about 0.2 to about 1.5, or from about 0.2 to about 1.0, or from about 0.2 to about 0.95, or from about 0.2 to about 0.90, or from about 0.2 to about 0.85, or from about 0.2 to about 0.80, or from about 0.2 to about 0.75, or from about 0.2 to about 0.70, or from about 0.2 to about 0.65, or from about 0.2 to about 0.60, or from about 0.2 to about 0.55, or from about 0.2 to about 0.50, or from about 0.2 to about 0.45, or from about 0.2 to about 0.40, or from about 0.2 to about 0.35, or from about 0.2 to about 0.30, or from about 0.2 to about 0.25, or from about 0.25 to about 3, or from about 0.25 to about 2.5, or from about 0.25 to about 2, or from about 0.25 to about 1.5, or from about 0.25 to about 1.0, or from about 0.25 to about 0.95, or from about 0.25 to about 0.90, or from about 0.25 to about 0.85, or from about 0.25 to about 0.80, or from about 0.25 to about 0.75, or from about 0.25 to about 0.70, or from about 0.25 to about 0.65, or from about 0.25 to about 0.60, or from about 0.25 to about 0.55, or from about 0.25 to about 0.50, or from about 0.25 to about 0.45, or from about 0.25 to about 0.40, or from about 0.25 to about 0.35, or from about 0.25 to about 0.30, or about 0.3 to about 3, or from about 0.3 to about 2.5, or from about 0.3 to about 2, or from about 0.3 to about 1.5, or from about 0.3 to about 1.0, or from about 0.3 to about 0.95, or from about 0.3 to about 0.90, or from about 0.3 to about 0.85, or from about 0.3 to about 0.80, or from about 0.3 to about 0.75, or from about 0.3 to about 0.70, or from about 0.3 to about 0.65, or from about 0.3 to about 0.60, or from about 0.3 to about 0.55, or from about 0.3 to about 0.50, or from about 0.3 to about 0.45, or from about 0.3 to about 0.40, or from about 0.3 to about 0.35, or about 0.35 to about 3, or from about 0.35 to about 2.5, or from about 0.35 to about 2, or from about 0.35 to about 1.5, or from about 0.35 to about 1.0, or from about 0.35 to about 0.95, or from about 0.35 to about 0.90, or from about 0.35 to about 0.85, or from about 0.35 to about 0.80, or from about 0.35 to about 0.75, or from about 0.35 to about 0.70, or from about 0.35 to about 0.65, or from about 0.35 to about 0.60, or from about 0.35 to about 0.55, or from about 0.35 to about 0.50, or from about 0.35 to about 0.45, or from about 0.35 to about 0.40, or about 0.4 to about 3, or from about 0.4 to about 2.5, or from about 0.4 to about 2, or from about 0.4 to about 1.5, or from about 0.4 to about 1.0, or from about 0.4 to about 0.95, or from about 0.4 to about 0.90, or from about 0.4 to about 0.85, or from about 0.4 to about 0.80, or from about 0.4 to about 0.75, or from about 0.4 to about 0.70, or from about 0.4 to about 0.65, or from about 0.4 to about 0.60, or from about 0.4 to about 0.55, or from about 0.4 to about 0.50, or from about 0.4 to about 0.45, about 0.45 to about 3, or from about 0.45 to about 2.5, or from about 0.45 to about 2, or from about 0.45 to about 1.5, or from about 0.45 to about 1.0, or from about 0.45 to about 0.95, or from about 0.45 to about 0.90, or from about 0.45 to about 0.85, or from about 0.45 to about 0.80, or from about 0.45 to about 0.75, or from about 0.45 to about 0.70, or from about 0.45 to about 0.65, or from about 0.45 to about 0.60, or from about 0.45 to about 0.55, or from about 0.45 to about 0.50, or about 0.5 to about 3, or from about 0.5 to about 2.5, or from about 0.5 to about 2, or from about 0.5 to about 1.5, or from about 0.5 to about 1.0, or from about 0.5 to about 0.95, or from about 0.5 to about 0.90, or from about 0.5 to about 0.85, or from about 0.5 to about 0.80, or from about 0.5 to about 0.75, or from about 0.5 to about 0.70, or from about 0.5 to about 0.65, or from about 0.5 to about 0.60, or from about 0.5 to about 0.55, or about 0.55 to about 3, or from about 0.55 to about 2.5, or from about 0.55 to about 2, or from about 0.55 to about 1.5, or from about 0.55 to about 1.0, or from about 0.55 to about 0.95, or from about 0.55 to about 0.90, or from about 0.55 to about 0.85, or from about 0.55 to about 0.80, or from about 0.55 to about 0.75, or from about 0.55 to about 0.70, or from about 0.55 to about 0.65, or from about 0.55 to about 0.60, or about 0.6 to about 3, or from about 0.6 to about 2.5, or from about 0.6 to about 2, or from about 0.6 to about 1.5, or from about 0.6 to about 1.0, or from about 0.6 to about 0.95, or from about 0.6 to about 0.90, or from about 0.6 to about 0.85, or from about 0.6 to about 0.80, or from about 0.6 to about 0.75, or from about 0.6 to about 0.70, or from about 0.6 to about 0.65, or about 0.65 to about 3, or from about 0.65 to about 2.5, or from about 0.65 to about 2, or from about 0.65 to about 1.5, or from about 0.65 to about 1.0, or from about 0.65 to about 0.95, or from about 0.65 to about 0.90, or from about 0.65 to about 0.85, or from about 0.65 to about 0.80, or from about 0.65 to about 0.75, or from about 0.65 to about 0.70, or about 0.7 to about 3, or from about 0.7 to about 2.5, or from about 0.7 to about 2, or from about 0.7 to about 1.5, or from about 0.7 to about 1.0, or from about 0.7 to about 0.95, or from about 0.7 to about 0.90, or from about 0.7 to about 0.85, or from about 0.7 to about 0.80, or from about 0.7 to about 0.75, or about 0.75 to about 3, or from about 0.75 to about 2.5, or from about 0.75 to about 2, or from about 0.75 to about 1.5, or from about 0.75 to about 1.0, or from about 0.75 to about 0.95, or from about 0.75 to about 0.90, or from about 0.75 to about 0.85, or from about 0.75 to about 0.80, or about 0.8 to about 3, or from about 0.8 to about 2.5, or from about 0.8 to about 2, or from about 0.8 to about 1.5, or from about 0.8 to about 1.0, or from about 0.8 to about 0.95, or from about 0.8 to about 0.90, or from about 0.8 to about 0.85, or about 0.85 to about 3, or from about 0.85 to about 2.5, or from about 0.85 to about 2, or from about 0.85 to about 1.5, or from about 0.85 to about 1.0, or from about 0.85 to about 0.95, or from about 0.85 to about 0.90, or about 0.9 to about 3, or from about 0.9 to about 2.5, or from about 0.9 to about 2, or from about 0.9 to about 1.5, or from about 0.9 to about 1.0, or from about 0.9 to about 0.95, or about 0.95 to about 3, or from about 0.95 to about 2.5, or from about 0.95 to about 2, or from about 0.95 to about 1.5, or from about 0.95 to about 1.0, or about 1.0 to about 3, or from about 1.0 to about 2.5, or from about 1.0 to about 2, or from about 1.0 to about 1.5, or about 1.5 to about 3, or from about 1.5 to about 2.5, or from about 1.5 to about 2, or about 2.0 to about 3, or from about 2.0 to about 2.5, or about 2.5 to about 3.

According to another embodiment, at least 30%, at least 35%, at least 40%, at least 45%, at least 50%, or at least 55%, or at least 60%, or at least 65%, or at least 70%, or at least 75%, or at least 80%, or at least 85%, or at least 90%, or at least 95% of the carboxyl groups are complexed with a multivalent cation. In embodiments, from about 30% to about 35%, or from about 30% to about 40%, or from about 30% to about 45%, or from about 30% to about 50%, or from about 30% to about 55%, or from about 30% to about 60%, or from about 30% to about 65%, or from about 30% to about 70%, or from about 30% to about 75%, or from about 30% to about 80%, or from about 30% to about 85%, or from about 30% to about 90%, or from about 30% to about 95%, or from about 35% to about 40%, or from about 35% to about 45%, or from about 35% to about 50%, or from about 35% to about 55%, or from about 35% to about 60%, or from about 35% to about 65%, or from about 35% to about 70%, or from about 35% to about 75%, or from about 35% to about 80%, or from about 35% to about 85%, or from about 35% to about 90%, or from about 35% to about 95%, or from about 40% to about 45%, or from about 40% to about 50%, or from about 40% to about 55%, or from about 40% to about 60%, or from about 40% to about 65%, or from about 40% to about 70%, or from about 40% to about 75%, or from about 40% to about 80%, or from about 40% to about 85%, or from about 40% to about 90%, or from about 40% to about 95%, or from about 45% to about 50%, or from about 45% to about 55%, or from about 45% to about 60%, or from about 45% to about 65%, or from about 45% to about 70%, or from about 45% to about 75%, or from about 45% to about 80%, or from about 45% to about 85%, or from about 45% to about 90%, or from about 45% to about 95%, or from about 50% to about 55%, or from about 50% to about 60%, or from about 50% to about 65%, or from about 50% to about 70%, or from about 50% to about 75%, or from about 50% to about 80%, or from about 50% to about 85%, or from about 50% to about 90%, or from about 50% to about 95%, or from about 55% to about 60%, or from about 55% to about 65%, or from about 55% to about 70%, or from about 55% to about 75%, or from about 55% to about 80%, or from about 55% to about 85%, or from about 55% to about 90%, or from about 55% to about 95%, or from about 60% to about 65%, or from about 60% to about 70%, or from about 60% to about 75%, or from about 60% to about 80%, or from about 60% to about 85%, or from about 60% to about 90%, or from about 60% to about 95%, or from about 65% to about 70%, or from about 65% to about 75%, or from about 65% to about 80%, or from about 65% to about 85%, or from about 65% to about 90%, or from about 65% to about 95%, or from about 70% to about 75%, or from about 70% to about 80%, or from about 70% to about 85%, or from about 70% to about 90%, or from about 70% to about 95%, or from about 75% to about 80%, or from about 75% to about 85%, or from about 75% to about 90%, or from about 75% to about 95%, or from about 80% to about 85%, or from about 80% to about 90%, or from about 80% to about 95%, or from about 85% to about 90%, or from about 85% to about 95%, or from about 85% to about 95%, of the carboxyl groups are complexed with a multivalent cation.

According to an embodiment, the preferred percentage of carboxyl groups complexed with a multivalent cation for the first carboxylated polymer having carboxyl groups is at least 30%.

According to another embodiment, the preferred percentage of carboxyl groups complexed with a multivalent cation for the second carboxylated polymer having carboxyl groups is at least 50%.

In the present invention, the carboxyl polymer preferably used to complex with multivalent cations is carboxymethyl-cellulose, due to several advantages:
inexpensive material and easy to obtain due to the large availability in the market;
physiologically inert and generally recognized as safe (GRAS) material;
compatible with a large number of API.

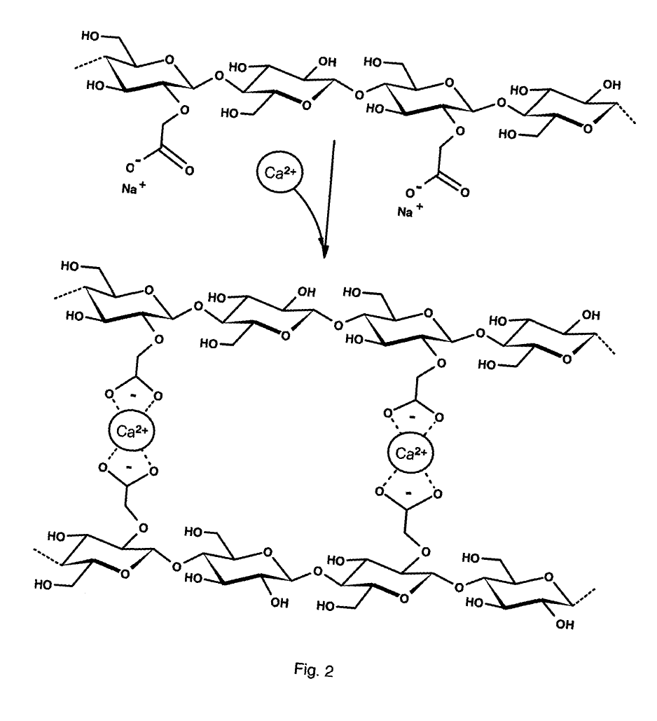

In a preferred embodiment, the complexation of carboxyl polymer with multivalent cations (i.e. calcium, FIG. 2) permit to eliminate or reduce anionic form as soluble sodium salts generating an insoluble complex more stable in gastrointestinal media. Although the complexation permit to obtain a stable matrix, it is not enough to control the release for highly soluble drugs and at higher dose such as metformin.

It is worth to mention that the matrix in the present invention is a monolithic tablet dosage form obtained simply by direct compression of the mixture of matrix and active principle powders.

To improve the stability or physicochemical properties and the control of the release of the highly soluble drug, the combination with other polymers is useful, such as control release polymers and second carboxylated polymer having carboxyl groups complexed with a divalent cation. This is a novelty for monolithic devices being widely different than that described previously for biphasic or multilayer dosage forms.

With the present invention, not only the release profiles are advantageous for sustained release, but the tablets are easier to obtain by direct compression, instead of multilayer devices.

For example methyl-cellulose, non-ionic cellulose ether obtained generally by treating cellulose in alkali medium with methyl chloride, is soluble only in cold water to form a colloidal solution. However, methyl-cellulose is insoluble and unable to swell in hot water. According to an embodiment methyl-cellulose may be incorporated with the calcium carboxyl polymer to confer to the matrix a hydrophobic character with stability at moderate high temperature including corporal temperature.

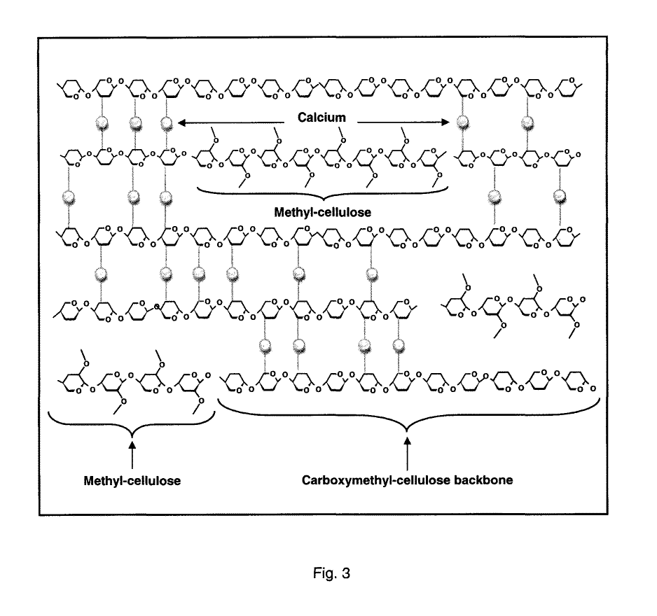

The incorporation can be done by entrapment and/or co-complexation which consists to introduce an appropriate quantity of methyl-cellulose (previously dissolved in cold water) in carboxymethyl-cellulose solution. The polymer mixture is mildly stirred at low temperature to favor stabilization mainly by hydrogen interactions. When the solution is homogenous, the addition of multivalent cations such as calcium allows triggering of the complexation reaction with carboxyl groups from carboxymethyl-cellulose (FIG. 3) and at the same time, entraps the methyl-cellulose into the Ca-Carboxymethyl-cellulose complex. Tablets obtained by this method are mechanically stable and no significant sticking or swelling in SGF at 37° C. is observed. A slight swelling is noticed in SIF with gel-like structure surrounding tablet. These properties are suitable to use as excipient for controlled release of highly soluble drugs such as metformin.

It is important to mention that a simple physical mixture of the powders did not give the same achievements the same results. Generally, tablets with physical mixture of methyl-cellulose and calcium carboxymethyl-cellulose complex are rapidly disintegrated in SGF. These results suggest rearrangements of interactions in the polymeric structure at the entrapment of methyl-cellulose into the calcium carboxymethyl-cellulose complex generating a more stable structure than that obtained by a physical mixture of the components.

Alternatively, ethyl-cellulose is a derivative of cellulose in which some of the hydroxyl groups are converted into ethyl ether groups. Ethyl-cellulose has a very low water up-take from air humidity or at in immersion and the small amount up-taken evaporates readily, leaving the ethyl-cellulose unaltered. To confer these properties to the matrix, ethyl-cellulose (previously dissolved in ethanol) can also be entrapped into the calcium carboxymethyl-cellulose complex, similarly as described for methyl-cellulose.

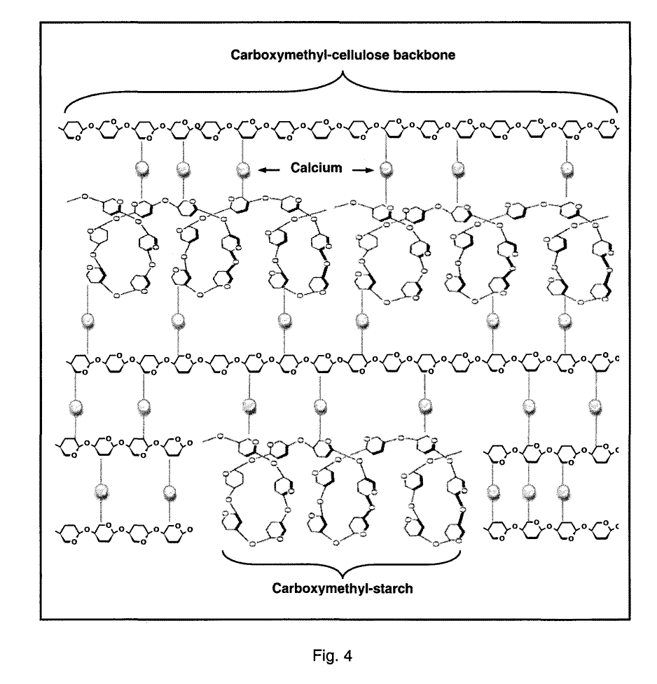

Another example is starch. The high amylose corn starch preferably used herein was Hylon VII provided by National Starch (Bridgewater, N.J., USA) with prominently a double helix B-type crystalline structure. After carboxymethylation, a new V-type single helix appeared. It is known that the starch helices are able to complex with other more or less hydrophobic molecules. There is an interest then to combine carboxymethyl-starch with carboxymethyl-cellulose and then stabilize by co-complexation with calcium (FIG. 4). This complex is believed useful as monolithic matrix for delayed release of insoluble or poorly soluble drugs such as Mesalamine, Diclofenac, Acetylsalicylic acid, etc.

A major achievement with this novel complexed excipient is that it can be useful to formulate both highly soluble or low soluble drugs. Sustained release with these types of drugs under monolithic tablet dosage form represents a novelty of this invention.

In a preferred embodiment, the carboxymethyl polymers are not limited to polysaccharides. For example, polymers such as polyacrylic acid (Carbomer) or copolymer methacrylate and acrylic acid (Eudragit) can be used in combination with other carboxyl polymers to generate the complex calcium carboxyl co-polymers.

In the present invention, carboxyl polymers are preferably carboxymethyl-cellulose, carboxymethyl starch or carboxymethyl high amylose starch, carboxyethyl starch or carboxyethyl high amylose starch, succinyl-starch or succinyl high amylose starch, carboxymethyl chitosan, carboxyethyl chitosan, succinyl chitosan, carboxymethyl guar gum, carboxymethyl hydroxypropyl guar gum, gellan gum, xanthan gum, alginate, pectate, hyaluronate, polyacrylic acid, polymethacrylic acid, copolymers of acrylic and methacrylic acids, etc. or combination thereof.

Other polymers that can be entrapped into the calcium carboxyl polymer complexes include but not limited to
cellulose (i.e. microcrystalline cellulose) and its derivatives such as methyl-cellulose, ethyl-cellulose, ethyl methyl-cellulose, hydroxyethyl-cellulose, hydroxyethyl methyl-cellulose, ethyl hydroxyethyl-cellulose, propyl-cellulose, hydroxypropyl-cellulose, hydroxypropyl methyl-cellulose, etc. or combination thereof;
starch and its derivatives such as hydroxypropyl-starch, starch acetate, cross-linked starch, etc. or combination thereof;
agar, agarose, guar, hydroxypropyl-guar, pullulan, carrageenan, scleroglucan, etc. or combination thereof;
polyvinyl-alcohol, polyethylene-glycol, polycaprolactone, polyvinyl-pyrrolidone, etc. or combination thereof.

According to an embodiment, the molecular weight of the above control release polymer and/or second carboxylated polymer having carboxyl groups complexed with a divalent cation is a molecular weight equal to or smaller than 200 kDa.

The multivalent cations used herein are preferably calcium. Other cations such as magnesium, zinc, aluminum, copper, etc. or combination thereof can be used for complexation.

In the preferred embodiment, a highly soluble drug is Metformin, and other high soluble drugs include without limitations Acyclovir, Alendronate, Atenolol, Bupropion, Captopril, Cinnarizine, Ciprofloxacin, Cisapride, Ganciclovir, G-CSF, Glipizide, Ketoprofen, Levodopa, Melatonin, Metoclopramide, Metoprolol, Minocyclin, Misoprostol, Nicardipine, Riboflavin, Sotalol, Tetracycline, Verapamil, etc.

In another embodiment other drugs having low solubility may be formulated and include without limitations Diclofenac, Sulfasalazine, Prednisone, Azathioprine, Metronidazole, Ampicillin, Ciprofloxacin, Cephalosporin, Furosemide, Tetracycline, Sulfonamide, Mesalamine, Acetylsalicylic acid, Irbesartan, Lisinopril, Rabeprazole, Sertraline, Simvastatin, Pioglitazone, Paroxetine, Terbinafine, Valproic, Venlafaxine, Atorvastatin, Bicalutamide, Citalopram, Fluoxetine, Supeudol, Pravastatin, Diltiazem, Bupropion, etc.

The present invention will be more readily understood by referring to the following examples which are given to illustrate the invention rather than to limit its scope.

### Example 1

### Preparation of the Complex <<Calcium Carboxymethyl-Cellulose/Methyl-Cellulose>> (Ca-CMC/MC): Entrapment of MC in CMC by Complexation with Calcium

### 1.1. Synthesis Ca-CMC/MC

An amount of 20 g of sodium carboxymethyl-cellulose (Aqualon®CMC extra-pure grade, DS 0.7-0.9, Wilmington, USA) is dispersed in 3.0 L of cold water under stirring. Thereafter, different amounts of methyl-cellulose (5-20 g in order to obtain various ratios) with different molecular weight (15-80 kDa) were each slowly introduced in a solution of CMC until obtaining a homogenous mixture. The complexation is done by adding an amount of calcium chloride about of 8% under stirring during at least 1.0 h. The complex calcium carboxymethyl-cellulose/methyl-cellulose complex is precipitated with an excess of acetone 80% and the precipitate is collected by decantation. The operation is repeated again and the precipitate is dried with pure acetone. The dry powder is finally obtained after air-drying or at 40° C. overnight in order to remove all traces of acetone solvent.

The powders can be alternatively obtained by using a spray-drying process which presents several advantages as fast, low cost and no solvent use.

### 1.2. Ca-CMC/MC Fourier Transform InfraRed (FTIR) Analysis

FTIR spectra were recorded on a Spectrum One (Perkin Elmer, Canada), instrument equipped with an UATR (Universal Attenuated Total Reflectance) device for samples in tablet (400 mg) form, in the spectral region (4000-650 cm⁻¹) with 24 scans/min at a 4 cm⁻¹ resolution. All spectra are corrected and normalized using the Spectrum software version 3.02.

The calcium carboxymethyl-cellulose/methyl-cellulose complex is investigated by comparing the FTIR spectra of MC, Na-CMC, Ca-CMC and Ca-CMC/MC tablets. The principle of method consists to highlight the level of hydration capacity of Ca-CMC/MC complex in comparison with the others materials before and after incubation for 2 h in SGF (pH 1.5).

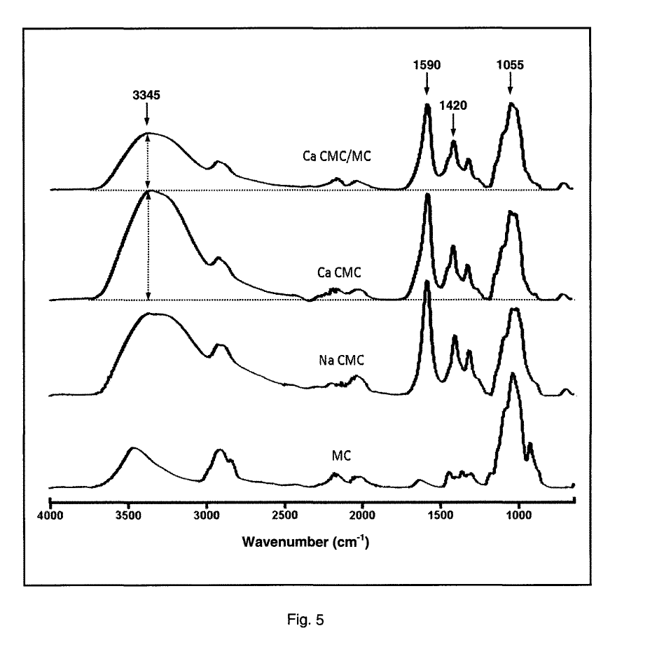

For Ca-CMC/MC (FIG. 5), the band at 3355 cm⁻¹ is mainly assigned to —O—H stretching vibration. The bands located at 1590 cm⁻¹ and 1420 cm⁻¹ are due to the —COO— asymmetric and symmetric stretches, respectively. The band at 1055 cm⁻¹ is attributed for —C—O bending.

The Na-CMC and Ca-CMC spectra (FIG. 5) showed absorption intensities near equal to those characteristic of the carboxylate. The symmetric and asymmetric stretching vibrations of the carboxylate are at positions similar to those of Ca-CMC/MC spectrum.

For Methyl-cellulose (FIG. 5), the bands located at 3410 cm⁻¹ and 1640 cm⁻¹ are assigned to —O—H stretching vibration, that at 1455 cm⁻¹ is ascribed to —C—CH and —O—CH bending, that at 1375 cm⁻¹ to —CH coupled with —OH bending and that at 1055 cm⁻¹ to —C—O bending vibration.

Although there are no significant differences between the FTIR spectrum of complex Ca-CMC/MC and those of Na-CMC and Ca-CMC, the band intensities of Ca-CMC/MC in the spectral region 3300-3400 cm⁻¹ (assigned mainly to the O—H stretching vibration, FIG. 5) are lower than those of Na CMC and Ca-CMC. This phenomenon indicates that there is a lower amount of bound water retained in the complex Ca-CMC/MC. This low water retention capacity seems related to the presence of methyl-cellulose which confers an insoluble character to the complex. In contrast, the intensity of wide O—H band in the spectral region 3300-3400 cm⁻¹ from Na CMC and Ca-CMC spectra is higher. These observations indicates that there is plenty of free-water retained suggesting that Na- and Ca-CMC possess a high capacity of hydration.

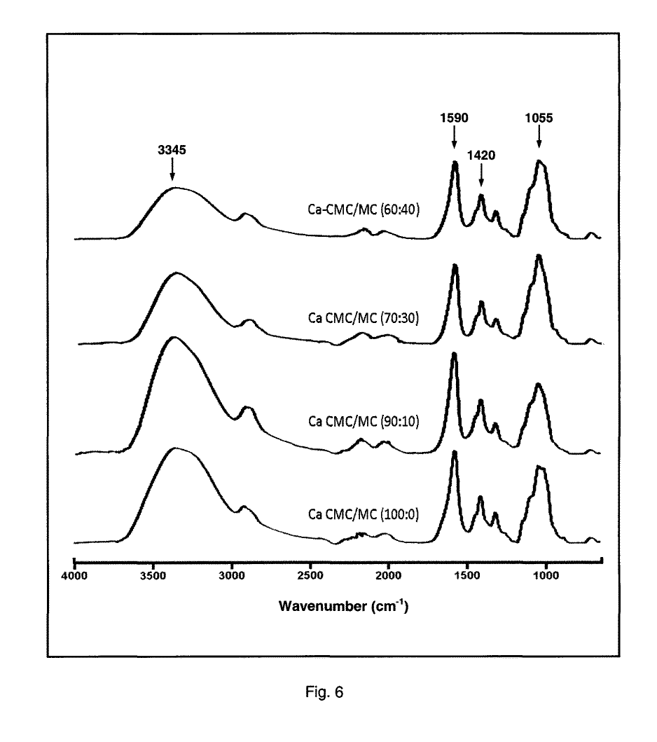

Similar observations for various ratios of MC entrapped in Ca-CMC are noticed. Indeed, at higher quantity of MC entrapped in Ca-CMC, lower free-water is retained in the tablet, as observed in FIG. 6.

Several changes in the infrared spectra are observed at low pH values compared to neutral or high pH. In SGF (pH 1.5), the protonation of the carboxyl is supported by the appearance of the new band at 1725 cm⁻¹ due to the carboxylic (—COOH) stretching vibrations. This is accompanied by a decrease in intensity or a disappearance of both the band at 1590 and 1420 cm⁻¹, ascribed to carboxylate (—COO⁻). The same changes of carboxyl group bands are observed for Ca-CMC.

The intensities of bands of Ca-CMC/MC in the spectral region 3300-3400 cm⁻¹ (assigned for O—H stretching vibrations) are lower than those of Ca-CMC, even at low pH values indicating that the hydration capacity of complex Ca-CMC/MC is low and independent of pH values in both SGF and SIF. This behavior is compatible with sustained release profiles.

### 1.3. In Vitro Dissolution Assay for Controlled Release of Metformin Using Ca-CMS/MC as Matrix

### 1.3.1. Preparation of Simulated Gastric Fluid (SGF, Pepsin-Free) at pH 1.5

The SGF is prepared according to United States Pharmacopeia (USP32-NF27). An amount of 2.0 g of sodium chloride and 7.0 mL of concentrate hydrochloric acid added in sufficient water to make 1 L. The pH value is about of 1.5.

### 1.3.2. Preparation of Simulated Intestinal Fluid (SIF, Pancreatin-Free) at pH 6.8

The SIF is prepared according to USP32-NF27. An amount of 6.8 g of monobasic potassium phosphate is dissolved in 250 mL water, and 77 mL of 0.2 M sodium hydroxide and 500 mL of water are added. The resulting solution is adjusted with either 0.2 M sodium hydroxide or 0.2 M hydrochloric acid to a pH of 6.8±0.1 and completed with water to 1 L.

### 1.3.3. Dissolution Assay

Two different formulations containing MC entrapped in CMC are use: Matrix 1 with ratio CMC/MC 60:40 and Matrix 2 with CMC/MC 70:30.

Monolithic tablets (biconvex oval-shaped) containing 500 mg of metformin hydrochloride and 330 mg of Matrix 1 or 2 are obtained by direct compression of powders (2.3 T/cm² in a Carver hydraulic press). Glumetza® tablet (500 mg) are used as reference (conventional form).

In vitro release of the tested formulations is carried out at 100 rpm and 37° C. using the Apparatus 2. The dissolution for Ca-CMC/MC tablets is first followed in simulated gastric fluid (SGF, pH 1.5) at 37° C. for 2 h and then the tablets are transferred in simulated intestinal fluid (SIF, pH 6.8) at 37° C. for 22 hours. As Glumetza® is based on the gastro-retentive technology, the dissolution assays with this dosage form are carried out only in SGF (pH 1.5).

A volume of 1 mL samples is withdrawn from the dissolution medium for each formulation (at intervals 0, 30, 60, 90 and 120 minutes for assay in SGF and every hour for assay in SIF). Each sample is properly diluted with the corresponding simulated fluids and filtered (0.20 μm). The Metformin concentration released from the tablets at each interval in 1 L of enzymes-free dissolution medium is measured spectrophotometrically at 233 nm for SGF and 250 nm for SIF. The release of Metformin is expressed as the relative percentage released at each time from the total amount of drug in each formulation.

### 1.3.4. Metformin Controlled Release Mechanism

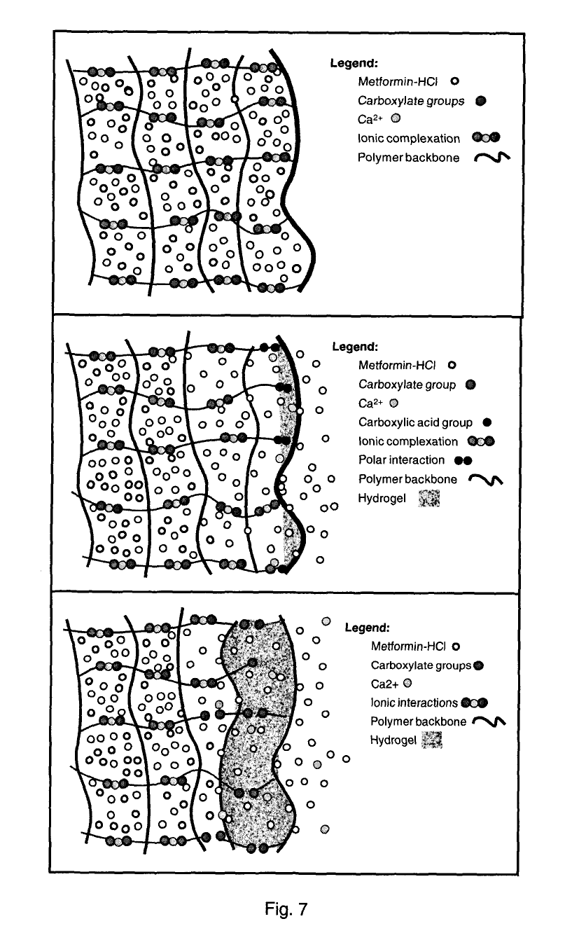

The release process of Metformin from the complex Ca-CMC/MC (FIG. 7) can be summarized as follow:

The CMC polymer chains are mainly stabilized by ionic interactions through complexation of carboxylate groups and calcium divalent cation, whereas MC is entrapped in the complex and stabilized by hydrogen association.

In the stomach (pH 1.5, at 37° C.), the hydration is limited due to the insoluble and less polar character of MC leading to a slow protonation of the carboxylate groups to carboxylic groups which occurs at the surface of the tablet after 2 h in SGF (ionic interactions are mainly changed to polar interactions). This phenomenon allows formation of a thinner gel-like structure surrounding the tablet and affording a continuous and slow release of a specific amount of Metformin in the stomach.

In the intestine (pH 6.8), a slow deprotonation of the outer carboxylic groups to carboxylate occurs, favoring external hydration which will stabilize the matrix mainly by hydrogen association evidenced by formation of a stable gel that control the sustained releases the remaining Metformin in the intestine.

### 1.3.5. Physical Characteristics of the Tablets of Ca-CMC/MC in SGF and SIF

The visual appearance of the tablets indicated that:

In SGF (incubation for 2 hours):
Glumetza® tablet is rapidly inflated and mainly in the height (axial) direction of the tablet at about two times its initial size. The tablet swelling is approximately 1.5 times in length and width (diagonal direction).
In contrast, Ca-CMC/MC maintained its shape with the formation of a thinner gel-like structure surrounding the tablet. The final tablet size showed a low increase considered as insufficient to be retained in the stomach, because their width and height are inferior to pylorus current opening of 14 mm in dogs (10 kg) and 30 mm in human (70 kg). This low swelling would allow the tablets to pass through the pylorus and reach the intestinal tract.

In SIF (after 2 h incubation in SGF):
For Ca-CMC/MC, the tablet size slightly increased after 4 h in SIF. After 6 h in SIF, no considerable change of the shape of the tablets are noticed, whereas the Glumetza® tablet shape is moderately altered to an almost rectangular shape, with edges slightly eroded. However, no evident reduction of the tablet volume is observed.
After 22 h of incubation in SIF, the tablets of Ca-CMC/MC are swollen and reached approximately 1.5-2.0 times their initial size. No evident change of shape is observed for these tablets during this interval. However, they formed a transparent semisolid, slightly sticky gel. For Glumetza® tablet, a reduction of size is observed, probably caused by erosion conferring to the tablet a round shape.

### 1.3.6. In Vitro Dissolution of Metformin Monolithic Tablets Using the Complex Ca-CMC/MC as Excipient

### According to the Ratios of CMC/MC

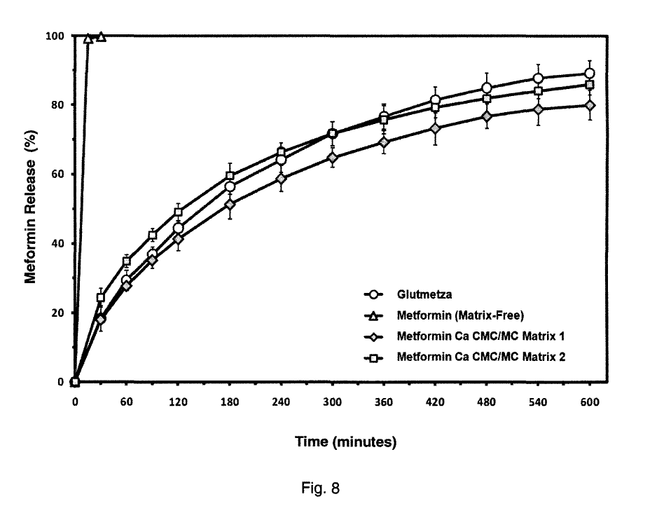

The dissolution profiles for the Ca-CMC/MC matrix-1 (ratio 60:40) and matrix-2 (70:30) versus Glumetza® commercial extended-release with the control (Metformin-HCl, matrix-free) are presented in FIG. 8.

The Metformin monolithic tablets formulated with the complex Ca-CMC/MC present similar in vitro kinetic profiles to those of Glumetza® extended-release form. A minor difference between matrix 1 and 2 are observed in SGF, but not in SIF.

### According to the Molecular Weight of MC Entrapped in Ca-CMC

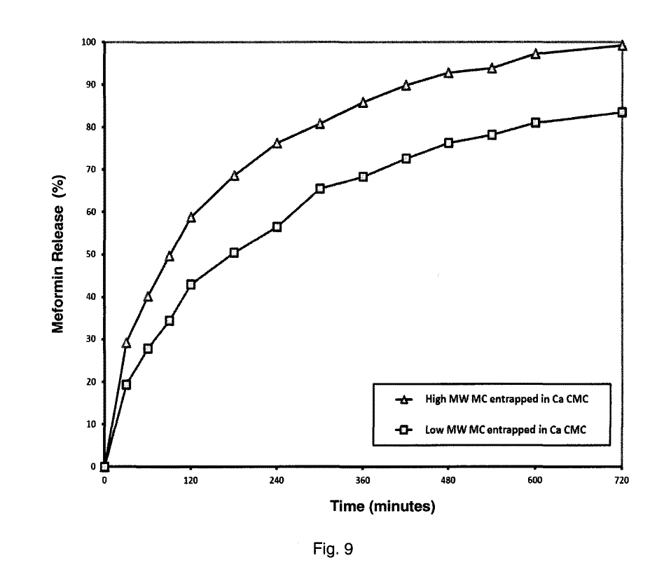

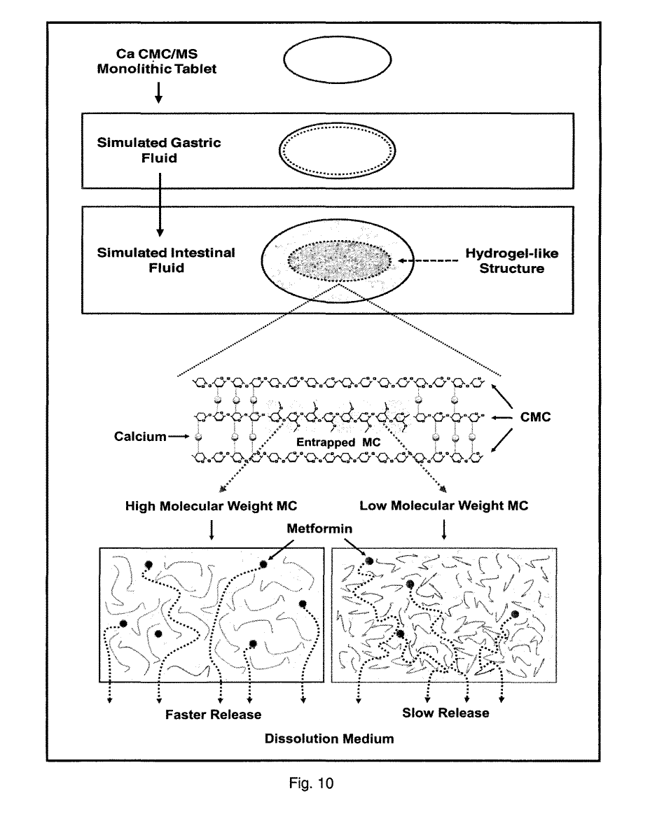

Metformin monolithic tablets containing a low molecular weight MC entrapped in CMC present a longer release compared to tablets with a high molecular weight MC (FIG. 9). This phenomenon is somewhat related to diffusion processes (FIG. 10). When the Ca-CMC/MC tablet reached the SIF, there is a hydration and swelling of tablet with formation of a gel-like structure which allows the controlled release of Metformin mainly by diffusion. However, the presence of entrapped MC in gel prevents this diffusion according to its molecular weight. At high molecular weight of MC, the Metformin easily and rapidly passes through the gel by diffusion. In contrast at low molecular weight of MC, the metformin spend more time inside the gel which leads a longer delivery time.

1.4. In Vivo Study on Dogs (Canis familiaris) for Metformin Controlled Release Using Ca-CMS/MC as Matrix

The main objective is to compare the pharmacokinetic parameters of the complex Ca-CMC/MC formulation of Metformin with those of a conventional form of metformin (Glumetza®) after oral administration.

### 1.4.1. Subjects and Study Design

This in vivo study is carried out on Beagle male dogs (Canis familiaris, average body weight 10.7±0.7 kg) and the experimental protocol is conducted according to the Animal Care Committee (INRS-Institut Armand-Frappier, Centre de Biologie Expérimentale, Laval, Québec, Canada) and is approved before experiment.

Upon each dog's receipt at the animal facility, the veterinarian evaluated the health condition of dogs and reviewed the medical records provided by the supplier animal facility (Marshall Bioresources, North Rose, N.Y.). To conform to the Animal Care Committee recommendations, the dogs are observed during one week for the acclimation period. Three groups (4 dogs) are subjected to treatment as follows:

Group-1: treated with monolithic tablets containing 500 mg of Metformin.HCl alone (Matrix-free);

Group-2: treated with monolithic tablets containing 500 mg Metformin.HCl and 330 mg complex Ca-CMC/MC Matrix-2 (ratio 60:40, Low Mw);

Group-3: treated with commercial Glumetza® extended release tablets.

After receiving treatments by oral administration, blood samples are collected in heparinized tubes (without anesthesia). In fact, an amount of 1.6 mL of blood samples are collected predose (t=0) and at the following postdose times: 0.5, 1.0, 1.5, 2.0, 2.5, 3.0, 3.5, 4.0, 6.0, 8.0, 10.0, 12.0 and 24 h. The metformin concentrations are determined by liquid chromatography with tandem mass spectrometry (LC-MS/MS) method (Heinig, K., Bucheli, F. 2004. J. Pharm. Biomed. Anal., 34, 1005-1011).

After blood sampling and centrifugation, the obtained plasma is submitted an extraction procedure as described by Tache et al. (Tache, F., David, V., Farca, A., Medvedovici, A. 2001. Microchem. J., 68, 13-19) which consists in using 4-nitrobenzoyl chloride to derivatize metformin (and metformin-d6 is used for internal standard). The derivatization is presented in Scheme 1.

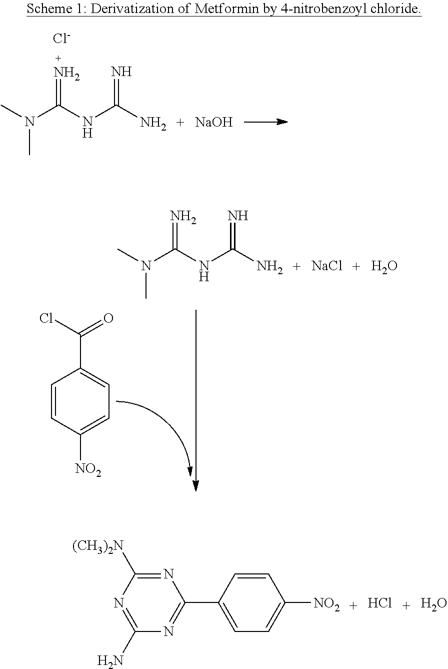

TABLE I
Pharmacokinetic parameters in Beagle dog of Metformin HydroChloride formulated
with Ca-CMC/MC complex and as commercial Glumetza ® tablets.

| | | Groups | | |
|---|---|---|---|---|
| Parameter | Unit | 1 | 2 | 3 |
| Test or control | | Metformin | Metformin monolithic | Glumetza ® |
| articles | | (Matrix-free) | tablet formulated with | (extended |
| | | | Ca-CMC/MC matrix | release GRDF) |
| Route of | | Oral | Oral | Oral |
| administration | | | | |
| Dose | mg | 500 | 500 | 500 |
| Number of | | 1 | 1 | 1 |
| doses | | | | |
| Cₘₐₓ | μg/mL | 12.4 | 4.8 | 3.9 |
| Tₘₐₓ | h | 1.3 | 2.0 | 2.5 |
| AUC_{0-24 h} | μg · h/mL | 77.9 | 32.3 | 30.3 |
| AUC_{0-∞} | μg · h/mL | 81.2 | 34.7 | 36.9 |
| T_{1/2} | h | 4.6 | 5.2 | 8.1 |
| Ke | 1/h | 0.157 | 0.133 | 0.086 |
| MRT | h | 8.02 | 8.0 | 13.2 |

Legend: GRDF = gastroretentive dosage form; AUC_{0-∞} = area under the concentration-time curve from time zero to infinity; AUC_{0-24 h} = area under the concentration-time curve from time zero to 24 hours; Cₘₐₓ = maximal concentration in plasma; Ke = terminal elimination rate per hour; MRT = mean residence time, T_{1/2} = elimination half-life, Tₘₐₓ = time (h) at maximal concentration.

Practically, an amount of 200 μL of each collected samples after centrifugation and 25 μL (5 μg/mL) of internal standard are placed in glass tubes. The derivatization occurs after introduction of 0.5 mL of 4-nitrobenzoyl chloride in dichloromethane (10 mg/mL) and 0.5 mL of NaOH 10%. The mixture is incubated during 1 h in the shaker (750 rpm) at room temperature and the extraction is performed with 4 mL of ethyl acetate. After vortexing, the organic phase (in the top) is transferred in small glass tubes and the organic extract is evaporated at 40° C. under a gentle stream of nitrogen. Finally, the dried material is reconstituted with 1 mL of mobile phase (methanol/acetonitrile/water, 6:1:3, v/v) with 10 mM of ammonium bicarbonate and submitted to liquid chromatography coupled to tandem mass spectrometry (LC-MS/MS) analysis.

The Metformin concentration in plasma extract is determined by the LC-MS/MS. The LC system includes component models: CBM-20A controller, DGU-14A and 20A online degassers, LC-10A DVP and LC-20AD pumps (Shimadzu, Tokyo, Japan) with a pre-column Zorbax Eclipse XDB-C8 (2.1×12.5 mm, 5 μm) and columns Zorbax SB-C18 (2.1×50 mm, 3.5 μm) and Zorbax XDB-C18 (3.0×150 mm, 5 μm; Agilent Technologies, CA, USA).

The chromatographic separation is achieved at room temperature using the mobile phase consisting of Methanol/Acetonitrile/Ammonium carbonate 10 mM (6:1:3 v/v) at a flow rate of 0.8 mL/min. The injection volume is varied 1-20 μL and the total run time cycle including equilibrium time is 4.0 minutes (3.0 min run time+1.0 min for injection). All solvents used are HPLC grade purchased from Fisher Co.

The standard curves of Metformin are prepared prior to each run and are generated using Metformin-d6 (C/D/N isotopes Inc., Qc, CA) as internal standard. For Mass Spectrophotometer, the model is API-4000 from Applied Biosystem (CA, USA). The MS fragments with best sensitivity for analysis had a mass/charge (m/z) ratio of 260.7 for derivatized Metformin and 266.8 for derivatized Metformin-d6; the parent m/z ratios are 215.2 and 221.2, respectively.

The pharmacokinetic parameters are calculated by using Thermo Kinetica™ software version 5.0. Metformin plasma concentration/times are analyzed using no compartmental pharmacokinetics to obtain parameters as follows:
peak plasma concentration (Cₘₐₓ);
time to reach the peak plasma concentration (Tₘₐₓ);
area under the concentration-time curve from time zero to last quantifiable concentration (AUC₀₋ₜ);
area under the concentration-time curve from time zero to infinity (AUC₀-∞);
elimination half-life (T_{1/2});
mean residence time (MRT).

Twelve dogs are randomly divided in groups of three, corresponding to the formulation group of the tablets. During the time-point sampling, each dog is observed for any signs of distress or excessive stress. Following these minor manipulations, all of the dogs are physically well and are clinically healthy after experiments.

### 1.4.2. Hematology and Urine Analysis

Blood sampling for hematology is taken at time 0 h predose and at 24.0 h postdose and a hematology assay including complete cell counts such as red (RBC) and white (WBC) blood cells; hemoglobin (Hb); hematocrit (Ht), mean corpuscular hemoglobin (MCH), reticulocytes and platelets.

Also, a differential WBC count (i.e. neutrophils, lymphocytes, monocytes, eosinophils, and basophils) and cells morphology (i.e., WBC, RBC, and platelets) is investigated for each sample. No abnormal signs are observed for each dog before and after experience.

Urine samples are also collected during the experience and analyzed with a Multistix® 10SG. The objective is to verify whether there are toxic signs after the experience. The urinary samples are taken before the exposure compared to that at 24.0 hours post-exposure. No differences are observed for these analyses.

The results of both hematology and urine analysis suggests no toxicity caused by the metformin formulations in the experiment can cause a toxicity.

### 1.4.2. In Vivo Results

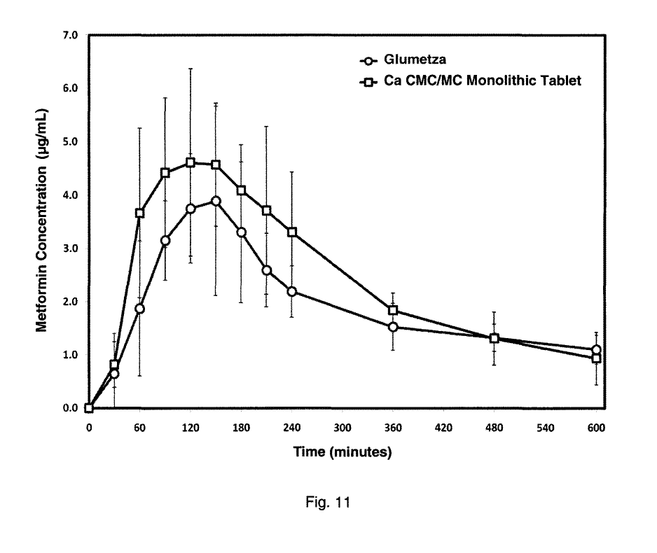

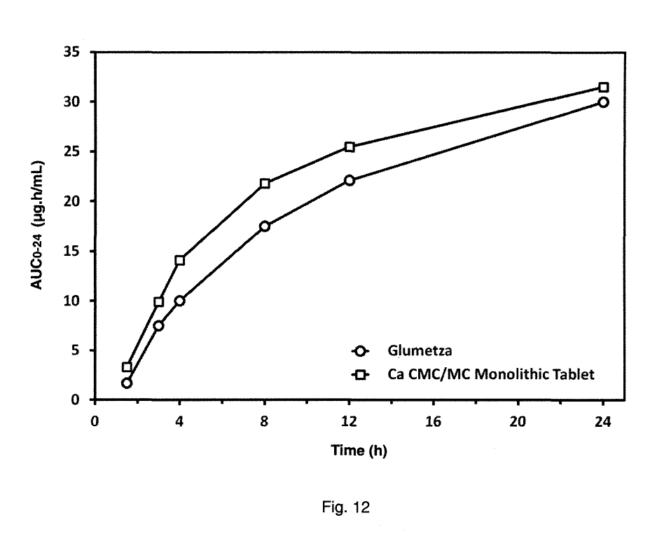

No significant difference in view of the pharmacokinetic profiles of metformin formulated with Ca-CMC/MC and Glumetza® (FIG. 11) are observed. Metformin hydrochloride pharmacokinetic parameters in Beagle dog of complex Ca-CMC/MC and commercial Glumetza® are presented in Table I and the Cumulative Area Under the Curve (AUC₀₋₂₄) in FIG. 12.

Similarly, other polysaccharides can be entrapped in CMC such as ethylcellulose, hydroxypropylcellulose, hydroxypropylmethylcellulose, etc. as described in the following examples.

### Example 2

### 2.1. Preparation of the Complex <<Calcium Carboxymethyl-Cellulose/Ethyl-Cellulose>> (Ca-CMC/EC) by Entrapment of EC in CMC and Complexation with Calcium

The preparation of Ca-CMC/EC is similarly as described in Example 1 (section 1.1.), with the variant that MC is replaced by EC previously dissolving in 200 mL of alcohol (preferably ethanol). Since EC is soluble in alcohol, the complex of Ca-CMG/EC powder can be preferably obtained by using a spray-drying process.

### 2.2. Preparation of the Complex <<Calcium Carboxymethylcellulose/Hydroxypropylcellulose>> (Ca-CMC/HPC) by Entrapment of HPC in CMC and Complexation with Calcium

The preparation of Ca-CMC/HPC is similarly as described in Example 1 (section 1.1.), with the variant that MC is replaced by HPC.

### 2.3. Preparation of the Complex <<Calcium Carboxymethylcellulose/Hydroxypropylmethylcellulose>> (Ca-CMC/HPMC) by Entrapment of HPMC in CMC and Complexation with Calcium

The preparation of Ca-CMC/HPMC is similarly as described in Example 1 (section 1.1.), with the variant that MC is replaced by HPMC.

### Example 3

### Preparation of the Complex Calcium Carboxymethyl-Cellulose/Carboxymethyl-Starch (Ca-CMC/CMS) by Co-Complexation of CMC/CMS with Calcium

### 3.1. Carboxymethylation of Starch

The carboxymethyl-starch is obtained by the reaction of starch in an alkaline solution with sodium monochloroacetate. An amount of 50 g of starch, preferably high amylose starch (Hylon VII, National Starch, NJ, USA), is gelatinized in 500 mL NaOH 3 M under stirring at room temperature until obtaining a homogenous suspension. After 1 h stirring at 40° C., an amount of 75 g of sodium monochloroacetate freshly dissolved in cold water are added to the starch suspension. The reaction is continued for at least 4 h at 60° C. After carboxymethylation, the mixture is neutralized (pH 7.2), precipitated in methanol and collected by filtration. The obtained residue is washed three times with pure methanol and dried in acetone to obtain the powders. It is of interest to note that the carboxymethyl-starch powder can alternatively be obtained by spray-drying.

### 3.2. Determination of Degree of Substitution of CMS

The degree of substitution is determined by titrimetric method as described by Le Tien et al. (2004, Biotechnol. Appl. Biochem., 39, 347-354) with modification as follows: the carboxyl groups of the carboxymethyl-starch (1.0 g) are first converted into the acidic (protonated) form by treatment of the modified polymer dispersed in ethanol with hydrogen chloride (1 M HCl). The protonated carboxymethyl-starch is then filtered, washed several times with ethanol/distilled water (80:20) in order to completely remove the acid in excess, and precipitated with pure acetone. Finally, a precise amount of carboxymethyl-starch is suspended in 100 mL distilled water. The acid form of the carboxymethyl-starch is titrated with a sodium hydroxide solution of known molarity (0.05 M).

Data obtained by titration showed that the number of carboxymethyl groups bound per glucose unit (degree of substitution, DS) may be >0.15. If the DS is <0.15, the subsequent carboxymethylation steps are run in a similar procedure described in the section 3.1 until obtaining a DS>0.15.

### 3.3. CMS FTIR Analysis

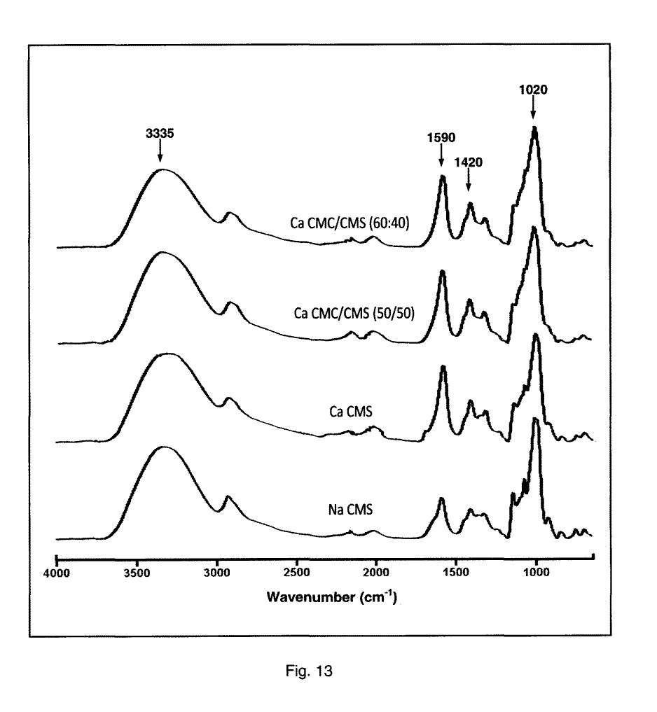

The FTIR analysis (FIG. 13) indicates the presence of carboxylate groups on obtained powders. After reaction, new absorption bands at 1590 and 1420 cm⁻¹ ascribed to carboxylate anions (asymmetric and symmetric stretching vibrations, respectively) confirmed starch carboxymethylation.

3.4. Synthesis of Ca-CMC/CMS by Co-Complexation of CMC/CMS with Calcium

An amount of 15 g of sodium carboxymethyl-cellulose (Aqualon®CMC extra-pure grade, DS 0.7-0.9, Wilmington, USA) is dispersed in 3.0 L of de-ionized water under stirring. Then, an amount of 15 g of sodium carboxymethyl-starch (obtaining as described in the section 3.1) is slowly introduced in the suspension until obtaining a homogenous mixture. The co-complexation with an amount of calcium chloride about of 8%) is continued under stirring for at least 1.0 h. The calcium carboxymethyl-cellulose/carboxymethyl starch complex is obtained by precipitation in excess of acetone 80% and the precipitate is collected by decantation. The operation is repeated again and the final precipitate is dried with pure acetone. The powder is finally obtained after air-dried or at 40° C. overnight in order to eliminate all traces of solvent. The powders can be alternatively obtained by spray-drying.

### 3.5. Ca-CMC/CMS FTIR Analysis

For Ca-CMC/CMS (FIG. 13), the band at 3335 cm⁻¹ is mainly assigned to —OH stretching vibration. Both bands located at 1590 cm⁻¹ and 1420 cm⁻¹ are ascribed to the —COO⁻ asymmetric and symmetric stretches, respectively whereas the band at 1020 cm⁻¹ is attributed for —C—O bending. Similar observations for Ca-CMS FTIR spectrum (FIG. 13) are noticed and no visible difference of absorption intensities is observed for carboxylate bands. In view of Na CMS FTIR spectrum, weak absorption intensities are observed for carboxylate bands.

Similar phenomenon observed with the Ca CMS/MC complex, where the absorption intensity assigned for O—H stretching vibrations for Ca-CMC/CMS (FIG. 13) is lower than those of Ca-CMS and Na CMS. This difference indicates a lesser water retention in the complex Ca-CMC/CMS than for the other forms. The explanation can be probably due to linear (CMC) and helical (CMS) structures forming the complex. This heterogeneous structure stabilized with calcium generates a tightly stable network, limiting thus the access of water inside the complex.

3.6. Dissolution Assay with the Monolithic Dosage Forms for Targeted Delivery of Mesalamine (5-Aminosalicylic Acid)

Monolithic tablets containing 400 mg of Mesalamine and 200 mg of complex Ca-CMS/MC are obtained by direct compression (2.3 T/cm² in a Carver hydraulic press).

In vitro assays are carried out at 100 rpm and 37° C. using the Apparatus 2. The dissolution of Mesalamine tablets formulated with Ca-CMC/MC complex is followed in simulated gastric fluid (SGF, pH 1.5) for 2 h and then the tablets are transferred in simulated intestinal fluid (SIF, pH 6.8) for 22 hours. A volume of 1 mL samples is withdrawn from the dissolution medium (at intervals 0, 30, 60, 90 and 120 minutes for assay in SGF and at each hour for assay in SIF), properly diluted with the corresponding simulated fluids and filtered (0.20 μm). The Mesalamine concentration released from the tablets in 1 L of enzymes-free dissolution medium (SGF or SIF) is measured spectrophotometrically at 300 nm for SGF and 330 nm for SIF. The release of Mesalamine is expressed as the relative percentage released at each time from the total amount of drug in each formulation.

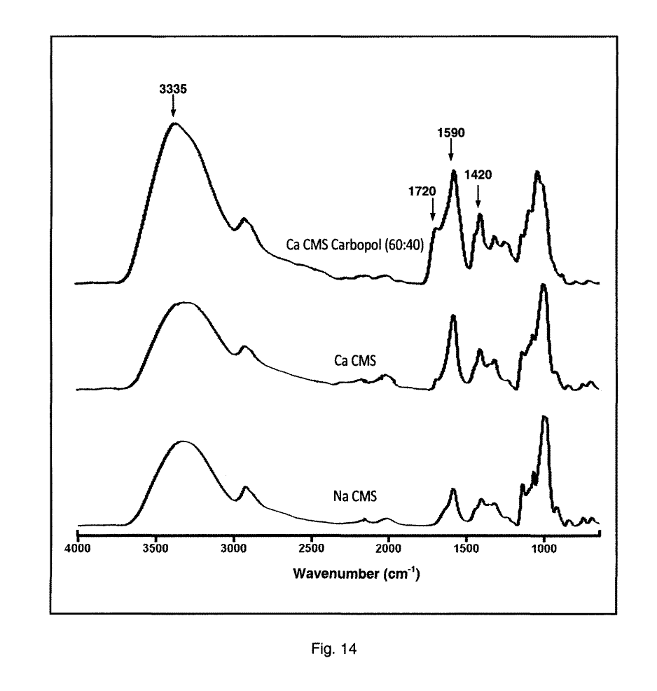

The proposed colon-targeted monolithic tablet form with the different Ca-carbohydrate complexes can ensure a gastro-protection by itself, eliminating the requirement of an expensive enteric coating. After 2 h in SGF, the tablets keep their structural integrity and less that 5% of Mesalamine is liberated due to a low hydration of the matrix in this acidic medium. After transfer in SIF, tablets hydrate slowly, resulting in a gradual swelling. The hydration control of the excipient in this neutral fluid manages the delivery. The active agent is liberated slowly over the first 3 h in SIF medium (FIG. 14). Then, the release rate of Mesalamine gradually increases to reach 60% after 10 h, with the complete liberation in 24 h.

### Example 4

### 4.1. Preparation of the Complex <<Calcium Carboxymethyl-Starch/Methyl-Cellulose>> (Ca-CMS/MC) by Entrapment of MC in CMS and Complexation with Calcium

In the preferred embodiment, not only Ca-CMC can be used to entrap MC, but CMS or other carboxyl polymers. Practically, an amount of 20 g of sodium carboxymethyl-starch (CMS) synthesized as described in the section 3.1 is dispersed in 3.0 L of cold water (˜10° C.) under stirring. Then, an amount of 20 g of methyl-cellulose is slowly introduced in the solution until obtaining a homogenous mixture. The complexation is done by adding an exceeding amount of calcium chloride at least 12% (w/w) under stirring for at least 1.0 h. The complex calcium carboxymethyl-starch/methyl-cellulose is obtained by precipitation in excess of acetone 80% and the precipitate is collected by decantation. The operation is repeated again and the precipitate is dried with pure acetone. The powder is finally obtained after air-drying or keeping at 40° C. overnight in order to remove traces of solvent. Alternatively, the powders can be obtained by spray-drying.

### 4.2. Preparation of the Complex <<Calcium Carboxymethyl-High Amylose Starch/Polyacrylic Acid>> (Ca-CMS/PAA) by Co-Complexation of CMS/PAA with Calcium

### 4.2.1. Synthesis

An amount of 30 g of sodium carboxymethyl-starch (obtained as described in the section 3.1) is dispersed in 3.0 L of cold water under stirring. Thereafter, an amount of 20 g of PAA (high molecular weight) is slowly introduced in the solution until obtaining a homogenous mixture. The complexation is starting by adding an amount of calcium chloride about of 12% under stirring for at least 1.0 h. The complex calcium carboxymethyl starch/polyacylic acid is obtained by precipitation in excess of pure acetone and the precipitate is collected by decantation. The operation is repeated again 3 or 4 times and the powder is finally obtained after air-dried or keeping at 40° C. overnight in order to eliminate traces of solvent. Alternatively, the powders can be obtained by spray-drying.

### 4.2.2. Ca-CMS/PAA FTIR Analysis

Ca-CMS/PAA FTIR spectrum (FIG. 14) shows a band at 3335 cm⁻¹ mainly assigned to —O—H stretching vibration. Both bands located at 1590 cm⁻¹ and 1420 cm⁻¹ are due to the carboxyl from CMS and PAA (—COO⁻ asymmetric and symmetric stretches, respectively). The band at 1055 cm⁻¹ is attributed for —C—O bending. Furthermore, a new prominent band located at 1720 cm⁻¹ is assigned for carboxylic acid, mainly from PAA.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A dosage form for delivery of an active ingredient comprising:
a first carboxylated polymer having carboxyl groups, having
a degree of substitution of at least 0.2,
a molecular weight of at least 200 kDa, and
at least 30% of said carboxyl groups being complexed with a divalent cation;
in a co-complex, entrapped within, or both, with at least one of
a control release polymer chosen from
an insoluble polymer or a polymer having a reduced water solubility at 30° C.,
a soluble polymer; and
a second carboxylated polymer having carboxyl groups complexed with a divalent cation.

2. The dosage form of claim 1, wherein said divalent cation is chosen from calcium, magnesium, zinc, aluminum, copper, or combinations thereof.

3. The dosage form of claim 1, wherein said divalent cation is calcium.

4. The dosage form of claim 1, wherein said control release polymer has a molecular weight equal to or smaller than the molecular weight of the first carboxylated polymer.

5. The dosage form of claim 1, wherein said second carboxylated polymer having carboxyl groups complexed with a divalent cation is having
a degree of substitution of at least 0.15,
a molecular weight equal to or smaller than the molecular weight of the first carboxylated polymer, and
at least 50% of said carboxyl groups being complexed with a divalent cation.

6. The dosage form of claim 1, wherein said first or second carboxylated polymer is chosen from a carboxymethylcellulose, a carboxymethyl starch, a carboxymethyl high amylose starch, a carboxyethyl starch, a carboxyethyl high amylose starch, a succinyl-starch, a succinyl high amylose starch, a carboxymethyl chitosan, a carboxyethyl chitosan, a succinyl chitosan, a carboxymethyl guar gum, a carboxymethyl hydroxypropyl guar gum, a gellan gum, a xanthan gum, a alginate, a pectate, a hyaluronate, a polyacrylic acid, a polymethacrylic acid, a copolymer of acrylic and methacrylic acids, or combination thereof.

7. The dosage form of claim 1, wherein said first carboxylated polymer is carboxymethylcellulose.

8. The dosage form of claim 1, wherein said second carboxylated polymer is carboxymethyl starch.

9. The dosage form of claim 1, wherein said insoluble polymer or polymer having a reduced water solubility at 30° C. is chosen from a cellulose, a methylcellulose, an ethylcellulose, an ethylmethylcellulose, an hydroxyethyl-cellulose, an hydroxyethylmethylcellulose, an ethyl hydroxyethylcellulose, a propylcellulose, an hydroxypropylcellulose, an hydroxypropylmethylcellulose, a starch, a hydroxypropylstarch, a starch acetate, a cross-linked starch, an agar, an agarose, a guar, an hydroxypropylguar, a pullulan, a carrageenan, a scleroglucan and combinations thereof.

10. The dosage form of claim 9, wherein said insoluble polymer or polymer having a reduced water solubility at 30° C. is a methylcellulose, an ethylcellulose, and an hydroxypropylmethylcellulose, or combinations thereof.

11. The dosage form of claim 1, wherein said soluble polymer is chosen from a polyvinylalcohol, a polyethyleneglycol, polycaprolactone, a polyvinyl-pyrrolidone, and combinations thereof.

12. The dosage form of claim 1, wherein a ratio of said first carboxylated polymer and said control release polymer, said second carboxylated polymer, or a combination thereof, is from about 1:1 to 90:10 w/w.

13. The dosage form of claim 12, wherein said ratio of said first carboxylated polymer and said control release polymer, said second carboxylated polymer, or a combination thereof, is about 60:40 w/w.

14. The dosage form of claim 12, wherein said ratio of said first carboxylated polymer and said control release polymer, said second carboxylated polymer, or a combination thereof, is about 70:30 w/w.

15. The dosage form of claim 12, wherein said ratio of said first carboxylated polymer and said control release polymer, said second carboxylated polymer, or a combination thereof, is about 90:10 w/w.

16. The dosage form of claim 12, wherein said ratio of said first carboxylated polymer and said control release polymer, said second carboxylated polymer, or a combination thereof, is about 1:1 w/w.

17. The dosage form of claim 1, wherein a molecular weight of said control release polymer or said second carboxylated polymer is from about 15 kDa to about 200 kDa.

18. The dosage form of claim 1, wherein a molecular weight of said control release polymer or said second carboxylated polymer is from about 15 kDa to about 80 kDa.

19. The dosage form of claim 1, wherein said degree of substitution of said first carboxylated polymer is from about 0.2 to about 2.

20. The dosage form of claim 1, wherein said degree of substitution of said first carboxylated polymer is from about 0.2 to about 0.9.

21. The dosage form of claim 1, wherein said degree of substitution of said first carboxylated polymer is from about 0.3 to about 0.9.

22. The dosage form of claim 1, wherein said degree of substitution of said first carboxylated polymer is from about 0.3 to about 0.7.

23. The dosage form of claim 1, wherein said degree of substitution of said first carboxylated polymer is from about 0.3 to about 0.5.

24. The dosage form of claim 1, wherein said degree of substitution of said first carboxylated polymer is about 0.5.

25. The dosage form of claim 1, wherein said degree of substitution of said second carboxylated polymer is from about 0.2 to about 2.

26. The dosage form of claim 1, wherein said degree of substitution of said second carboxylated polymer is from about 0.2 to about 1.

27. The dosage form of claim 1, wherein said degree of substitution of said second carboxylated polymer is from about 0.3 to about 0.9.

28. The dosage form of claim 1, wherein said degree of substitution of said second carboxylated polymer is from about 0.3 to about 0.7.

29. The dosage form of claim 1, wherein said degree of substitution of said second carboxylated polymer is from about 0.3 to about 0.5.

30. The dosage form of claim 1, wherein said degree of substitution of said second carboxylated polymer is about 0.5.

31. The dosage form of claim 1, wherein said first carboxylated polymer is carboxymethyl cellulose having degree of substitution of about 0.5.

32. The dosage form of claim 1, wherein said first carboxylated polymer is carboxymethyl starch having degree of substitution of about 0.5.

33. The dosage form of any one of claims 1 to 33, further comprising said active ingredient.

34. The dosage form of claim 33, wherein said active ingredient is chosen from a highly soluble drug, or a drug having low solubility.

35. The dosage form of claim 34, wherein said highly soluble drug is chosen from metformin, acyclovir, alendronate, atenolol, bupropion, captopril, cinnarizine, ciprofloxacin, cisapride, ganciclovir, g-csf, glipizide, ketoprofen, levodopa, melatonin, metoclopramide, metoprolol, minocyclin, misoprostol, nicardipine, riboflavin, sotalol, tetracycline, and verapamil.

36. The dosage form of claim 34, wherein said highly soluble drug is metformin.

37. The dosage form of claim 34, wherein said drug having low solubility is chosen from diclofenac, sulfasalazine, prednisone, azathioprine, metronidazole, ampicillin, ciprofloxacin, cephalosporin, furosemide, tetracycline, sulfonamide, mesalamine, acetylsalicylic acid, irbesartan, lisinopril, rabeprazole, sertraline, simvastatin, pioglitazone, paroxetine, terbinafine, valproic, venlafaxine, atorvastatin, bicalutamide, citalopram, fluoxetine, supeudol, pravastatin, diltiazem, and bupropion.

38. The dosage form of claim 34, wherein said drug having low solubility is mesalamine.

37. The dosage form of claim 34, wherein said highly soluble drug is from about 500 mg to about 1200 mg metformin, said first carboxylated polymer having carboxyl groups is carboxymethyl cellulose and said control release polymer is methylcellulose.

39. The dosage form of claim 34, wherein said drug having low solubility is from about mg 400 to about 1000 mg mesalamine, said first carboxylated polymer having carboxyl groups is carboxymethyl cellulose and said control release polymer is methylcellulose.

40. A method of treating diabetes comprising administering to a subject in need thereof a dosage form of claim 39.

41. A method of treating an inflammatory bowel disease comprising administering to a subject in need thereof a dosage form of claim 39.

42. Use of the dosage form of claim 39 for treating diabetes.

43. Use of the dosage form of claim 39 for treating an inflammatory bowel disease.

44. The dosage form of claim 39 for use in the treatment of diabetes.

45. The dosage form of claim 39 for use in the treatment of an inflammatory bowel disease.

46. A process for the preparation of a carboxylated polymer having carboxyl groups complexed with a divalent cation comprising:
a) in a solution at pH>5.5, contacting a carboxylated polymer having carboxyl groups, having a degree of substitution of at least 0.2, and a molecular weight of at least 200 kDa with a source of divalent cation for a time sufficient for at least 50% of said carboxyl groups to be complexed with said divalent cation.

47. The process of claim 46, further comprising step b):
b) precipitating said carboxylated polymer having carboxyl groups complexed with a divalent cation, to obtain a precipitated carboxylated polymer having carboxyl groups complexed with a divalent cation.

48. The process of claim 47, further comprising step c):
b) collecting and drying said precipitated carboxylated polymer having carboxyl groups complexed with a divalent cation.

49. The process of any one of claims 46 to 48, wherein said source of divalent cation is calcium chloride, calcium lactate, calcium acetate, calcium gluconate, and combinations thereof.

50. A process for the preparation of an inclusion complex, a co-complex, or both comprising:
a) contacting a solution at pH>5.5, containing
a first carboxylated polymer having carboxyl groups, having a degree of substitution of at least 0.2, and a molecular weight of at least 200 kDa, and
at least one of
a control release polymer chosen from
an insoluble polymer or a polymer having a reduced water solubility at 30° C.,
a soluble polymer; and
a second carboxylated polymer having carboxyl groups complexed with a divalent cation.
with a source of divalent cation for a time sufficient for at least 50% of said carboxyl groups to be complexed with said divalent cation.

51. The process of claim 50, wherein said divalent cation is chosen from calcium, magnesium, zinc, aluminum, copper, or combinations thereof.

52. The process of claim 50, wherein said divalent cation is calcium.

53. The process of claim 50, wherein said second carboxylated polymer having carboxyl groups complexed with a divalent cation is having
a degree of substitution of at least 0.15,
a molecular weight equal to or smaller than the molecular weight of the first carboxylated polymer, and
at least 50% of said carboxyl groups being complexed with a divalent cation.

54. The process of claim 50, wherein said first or second carboxylated polymer is chosen from a carboxymethylcellulose, a carboxymethyl starch, a carboxymethyl high amylose starch, a carboxyethyl starch, a carboxyethyl high amylose starch, a succinyl-starch, a succinyl high amylose starch, a carboxymethyl chitosan, a carboxyethyl chitosan, a succinyl chitosan, a carboxymethyl guar gum, a carboxymethyl hydroxypropyl guar gum, a gellan gum, a xanthan gum, a alginate, a pectate, a hyaluronate, a polyacrylic acid, a polymethacrylic acid, a copolymer of acrylic and methacrylic acids, or combination thereof.

55. The process of claim 50, wherein said first carboxylated polymer is carboxymethylcellulose.

56. The process of claim 50, wherein said second carboxylated polymer is carboxymethyl starch.

57. The process of claim 50, wherein said insoluble polymer or polymer having a reduced water solubility at 30° C. is chosen from a cellulose, a methylcellulose, an ethylcellulose, an ethylmethylcellulose, an hydroxyethyl-cellulose, an hydroxyethylmethylcellulose, an ethyl hydroxyethylcellulose, a propylcellulose, an hydroxypropylcellulose, an hydroxypropylmethylcellulose, a starch, a hydroxypropylstarch, a starch acetate, a cross-linked starch, an agar, an agarose, a guar gum, an hydroxypropylguar, a pullulan, a carrageenan, a scleroglucan and combinations thereof.

58. The process of claim 57, wherein said insoluble polymer or polymer having a reduced water solubility at 30° C. is a methylcellulose, an ethylcellulose, and an hydroxypropylmethylcellulose, or combinations thereof.

59. The process of claim 50, wherein said soluble polymer is chosen from a polyvinylalcohol, a polyethyleneglycol, polycaprolactone, a polyvinyl-pyrrolidone, and combinations thereof.

60. The process of claim 50, wherein a ratio of said first carboxylated polymer and said control release polymer, said second carboxylated polymer, or a combination thereof, is from about 1:1 to 90:10 w/w.

61. The process of claim 60, wherein said ratio of said first carboxylated polymer and said control release polymer, said second carboxylated polymer, or a combination thereof, is about 60:40 w/w.

62. The process of claim 60, wherein said ratio of said first carboxylated polymer and said control release polymer, said second carboxylated polymer, or a combination thereof, is about 70:30 w/w.

63. The process of claim 60, wherein said ratio of said first carboxylated polymer and said control release polymer, said second carboxylated polymer, or a combination thereof, is about 90:10 w/w.

64. The process of claim 60, wherein said ratio of said first carboxylated polymer and said control release polymer, said second carboxylated polymer, or a combination thereof, is about 1:1 w/w.

65. The process of claim 50, wherein a molecular weight of said control release polymer or said second carboxylated polymer is from about 15 kDa to about 200 kDa.

66. The process of claim 50, wherein a molecular weight of said control release polymer or said second carboxylated polymer is from about 15 kDa to about 80 kDa.

67. The process of claim 50, wherein said degree of substitution of said first carboxylated polymer is from about 0.2 to about 2.

68. The process of claim 50, wherein said degree of substitution of said first carboxylated polymer is from about 0.2 to about 0.9.

69. The process of claim 50, wherein said degree of substitution of said first carboxylated polymer is from about 0.3 to about 0.9.

70. The process of claim 50, wherein said degree of substitution of said first carboxylated polymer is from about 0.3 to about 0.7.

71. The process of claim 50, wherein said degree of substitution of said first carboxylated polymer is from about 0.3 to about 0.5.

72. The process of claim 50, wherein said degree of substitution of said first carboxylated polymer is about 0.5.

73. The process of claim 50, wherein said degree of substitution of said second carboxylated polymer is from about 0.2 to about 1.0.

74. The process of claim 50, wherein said degree of substitution of said second carboxylated polymer is from about 0.2 to about 0.9.

75. The process of claim 50, wherein said degree of substitution of said second carboxylated polymer is from about 0.3 to about 0.9.

76. The process of claim 50, wherein said degree of substitution of said second carboxylated polymer is from about 0.3 to about 0.7.

77. The process of claim 50, wherein said degree of substitution of said second carboxylated polymer is from about 0.3 to about 0.5.

78. The process of claim 50, wherein said degree of substitution of said second carboxylated polymer is about 0.5.

79. The process of claim 50, wherein said first carboxylated polymer is carboxymethyl cellulose having degree of substitution of about 0.5.

80. The process of claim 50, wherein said first carboxylated polymer is carboxymethyl starch having degree of substitution of about 0.5.

81. The process of claim 50, further comprising step b):
b) precipitating said inclusion complex, said co-complex, or both, to obtain a precipitated inclusion complex, a precipitated co-complex, or both.

82. The process of claim 47, further comprising step c):
c) collecting and drying said precipitated inclusion complex, said co-complex, or both.
